(19) United States
(12) Patent Application Publication
(10) Pub. No.: US 2025/0268945 A1
(43) Pub. Date: Aug. 28, 2025

(54) COMPOSITIONS AND METHODS FOR TREATING OSTEOARTHRITIS

(71) Applicant: BRITECYTE INC., Frederick, MD (US)

(72) Inventor: Alla Danilkovitch, Columbia, MD (US)

(21) Appl. No.: 19/060,021

(22) Filed: Feb. 21, 2025

Related U.S. Application Data
(60) Provisional application No. 63/557,122, filed on Feb. 23, 2024.

(51) Int. Cl. A61K 35/35; A61K 9/00; A61K 35/28; A61P 19/02

(52) U.S. Cl. CPC A61K 35/35; A61K 9/0019; A61K 35/28; A61P 19/02

(57) ABSTRACT

Disclosed are methods of treating a subject having osteoarthritis comprising administering to the subject a composition, wherein the composition comprises an adipose matrix comprising devitalized and partially delipidized adipose tissue. Disclosed are methods of treating or ameliorating a symptom of osteoarthritis in a subject comprising: administering to the subject a composition, wherein the composition comprises an adipose matrix comprising devitalized and partially delipidized adipose tissue. Disclosed are methods of inhibiting inflammatory cytokines in a subject having osteoarthritis comprising administering to the subject a composition, wherein the composition comprises an adipose matrix comprising devitalized and partially delipidized adipose tissue. Disclosed are methods of inhibiting MMP-13 in a subject having osteoarthritis comprising administering to the subject a composition, wherein the composition comprises an adipose matrix comprising devitalized and partially delipidized adipose tissue.

### CROSS-REFERENCE TO RELATED APPLICATIONS

This application claims the benefit of U.S. Provisional Patent Application No. 63/557,122, filed Feb. 23, 2024, which is incorporated by reference herein in its entirety.

### BACKGROUND

Osteoarthritis (OA) is a degenerative joint disease characterized by joint pain due to inflammation, which results in the breakdown of joint cartilage. Previously, it was proposed that OA is a “wear-and-tear” joint disease due to inflammation and mechanical stress of excessive weight on joints. Although excessive weight is a risk factor for the disease, OA also occurs in non-weight bearing joints, and weight loss does not relieve symptoms. The positive association between obesity and OA is reported not only for knee joints but also for non-weight bearing joints, such as hands. Non-weight bearing joint involvement in OA suggests that joint damage may be caused by systemic factors of the adipose tissue, called adipokines, which provide a metabolic link between obesity and OA. Indeed, there is a correlation between plasma and synovial levels of adiponectin, one of the key adipokines in the adipose tissue, and severity of OA. These findings suggest that OA is linked to metabolic dysfunction of adipose in the body and less to mechanical joint overloading.

Historically, adipose tissue was considered an inert filler. Today it is recognized that adipose tissue has regenerative properties including anti-inflammatory activity. Recent data strengthen the hypothesis that OA is a systemic disorder in which dysregulation of lipid homeostasis can be one of the pathophysiological mechanisms leading to OA. Currently, the use of autologous adipose tissue intraarticular injections has emerged as a new therapeutic approach for the treatment of OA. The regenerative potential of adipose tissue is attributed to the high content of mesenchymal stromal stem cells (Adipose Derived Stem Cells, ADSCs) located in the Stromal Vascular Fraction (SVF) of the tissue. ADSCs are progenitor cells with multilinear plasticity towards adipo-, chondro-, and osteo-differentiation and with tissue regenerative, immunomodulatory, and pro-angiogenic properties. Thus, it is believed that the use of autologous adipose transplantation for OA will allow not only the shutdown of inflammation, but also may lead to regeneration of articular cartilage by ADSCs present in the adipose tissue. The protocols for harvesting, processing, and reinjection of autologous subcutaneous adipose are well established, and data in the literature report positive clinical outcomes after intra-articular injections of autologous adipose: a significant decrease in the patient's symptomatic discomfort, with improvement in joint function and reduction in pain.

### BRIEF SUMMARY

BRC001, an innovative regenerative medicine technology targeting diseases with adipose tissue dysfunction including non-alcoholic steatohepatitis (NASH), is currently being developed. Adipose tissue is recognized as the largest endocrine organ in the body, regulating metabolism, inflammation, and tissue regeneration. Adipose dysfunction contributes to multi-organ damage and is involved in the pathogenesis of many diseases. BRC001 is an off-the-shelf allogeneic engineered adipose therapy composed of a combination of allogeneic adipose-derived SVF cells (MSCs) and the partially delipidized devitalized adipose tissue. MSCs may act via a paracrine secretion of growth factors and via differentiation in adipocytes. The adipose tissue is an essential component in BRC001 that serves as a carrier for the stem cells and provides an adipose-specific microenvironment to support implanted MSCs and host cell functionality. The BRC001 formulation is a platform technology that has a broad therapeutic potential to treat many inflammatory diseases and conditions including OA. However, characterization of adipose tissue and MSCs, the two components of the BRC001 formulation, led to unexpected findings. The results discussed herein demonstrate that the adipose tissue, and not adipose-derived stem cells specifically, possesses the majority of anti-inflammatory activity. In addition, the tissue is a source of adipokines that can help to maintain a proper metabolic microenvironment in the joints to protect from the development and progression of OA. These results indicate that the compositions described herein having devitalized and partially delipidized adipose tissue (e.g. BRC001 adipose tissue component alone (no cells)) can be used for the treatment of inflammatory diseases including OA. Thus, in some aspects, the disclosed compositions are similar to BRC001 except the disclosed compositions do not necessarily comprise MSCs. These findings are the opposite of the current opinion that stem cells in adipose are required for adipose tissue to have therapeutic effects. Another current thinking is that to be able to use adipose tissue, lipids should be removed from the tissue (delipidization). In contrary, the disclosed experimental data demonstrate therapeutic benefits of retaining lipids in the tissue. Delipidization of the adipose tissue results in significant decrease of anti-inflammatory activity of the tissue, which is an essential characteristic for the successful treatment of OA. Examples presented below support that adipose tissue without viable cells has biological activities and factors required for successful OA treatment.

Disclosed are methods of treating a subject having osteoarthritis comprising administering to the subject a composition, wherein the composition comprises an adipose matrix comprising devitalized and partially delipidized adipose tissue.

Disclosed are methods of treating or ameliorating a symptom of osteoarthritis in a subject comprising administering to the subject a composition, wherein the composition comprises an adipose matrix comprising devitalized and partially delipidized adipose tissue.

Disclosed are methods of inhibiting inflammatory cytokines in a subject having osteoarthritis comprising administering to the subject a composition, wherein the composition comprises an adipose matrix comprising devitalized and partially delipidized adipose tissue.

Disclosed are methods of inhibiting MMP-13 in a subject having osteoarthritis comprising administering to the subject a composition, wherein the composition comprises an adipose matrix comprising devitalized and partially delipidized adipose tissue.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic of BRC001 processing, components, fabrication and characterization.

FIGS. 2A and 2B show (A) morphology and (B) phenotype of isolated and culture expanded adipose-derived SVF cells. The cell phenotype was evaluated at passage 2 (P2) by Fluorescence Activated Cell Sorting (FACS). The cells are >96% positive (the lowest % among 10 tested donors) for mesenchymal stem cell (MSC) markers CD73 and CD90, and negative (<1%, the highest % among 10 tested donors) for CD45, CD34, and CD105 hematopoietic stem cell (HST) markers. Representative FACS histograms for one out of 10 tested donors shown with >99% CD73 and CD90 positive cells and <0.02% CD45, CD34, and CD105 positive cells.

FIG. 3 shows microscopic images of adipo-differentiated adipose-derived SVF cells at P2. Cells were cultured in a 24-well plate for 7 days in an adipo-differentiation medium. Then, cells were fixed, and differentiated cells were visualized by staining of intracellular lipids with Oil Red O. Results of adipo-differentiation of cells from 2 donors with the highest and the lowest number of differentiated cells are shown.

FIG. 4 shows that the structure of native adipose tissue retained in the BRC001 tissue component. Histological images of H&E-stained tissue sections at 20× magnification are shown.

FIG. 5 shows concentrations of inflammatory cytokines in BRC001 tissue and cell components. Inflammatory cytokines were measured in cell-derived culture supernatants and tissue extracts using a multiplex immunoassay. Results for 10 donors are presented as a heat map.

FIG. 6 shows concentrations for 19 cytokines and growth factors in the BRC001tissue component. Cytokines and growth factors were measured in tissue extracts using a multiplex immunoassay. Results for 10 donors are presented as a heat map.

FIG. 7 shows concentrations for 19 cytokines and growth factors in the BRC001 cell component. Cytokines and growth factors were measured in cell-derived culture supernatants using a multiplex immunoassay. Results for 10 donors are presented as a heat map.

FIGS. 8A-8C show that: (A) BRC001 cell component is the source of angiogenic factors (Vascular Endothelial Growth Factor—VEGF), required for new blood vessel formation (angiogenesis); (B) BRC001 tissue component is a reservoir of adipose-specific metabolic factors (i.e., adiponectin), and (C) BRC001 tissue component, and not cells, is the major source of anti-inflammatory activity. Bars are mean+/−SD for 10 donors.

FIG. 9 shows FACS histograms of human CD90 stained cells that were isolated from BRC001 (tissue+cells, top panel) and BRC001 tissue component only (bottom panel) grafts at different study days post-implantation in vivo. Positive control: human adipose-derived culture expanded SVF cells from the BRC001 donor. Day 0 sample: cells isolated from BRC001 prior to implantation. Pink colored histograms—isotype control; white colored histograms—human CD90+ stained cells.

FIG. 10 shows NUMA-1 positively stained human cells in BRC001 post-implantation in vivo. Human cells were detectable in BRC001 only on Day 0 prior to implantation, and on Days 3 and 7 post-implantations. Red arrows point to NUMA-1 positively stained human cells (brown colored on the images).

FIG. 11 shows persistence of BRC001 tissue component (triangles) and BRC001 cell component (circles) in vivo. BRC001 tissue component alone or BRC001 composed of the tissue and cells were implanted subcutaneously in SD rats. At different time points, implanted formulations were identified and excised. BRC001 tissue explants were weighed, and the tissue weight is presented on the graph as % of the BRC001 tissue weight on day 0. Weight of the BRC001 tissue component on day 0 is considered 100%. Variability of the BRC001 tissue weight between time points is due to the presence of host tissues fused with the implants, and therefore it was not possible to dissect out the host tissue. The presence of viable human cells in BRC001 after implantation was evaluated by FACS. Cells were isolated from BRC001 explants by enzymatic digestion. FACS analysis for CD90+ human cells was used to detect human cells in the BRC001 explants. Number of detected cells is expressed in % from day 0. Number of the BRC cells at day 0 is considered 100%.

FIG. 12 shows a schematic of BRC-OA formulation (formerly BRC001 tissue component) and its characterization.

FIG. 13 shows that BRC-OA anti-inflammatory activity is mediated by high and low molecular weight soluble factors present in the tissues. LPS-stimulated THP-1 cells (a monocyte/macrophage model) were co-cultured with 3 different concentrations of the BRC-OA tissue or un-fractioned or fractioned BRC-OA tissue extracts. TNF-□ secreted by LPS-stimulated THP-1 cells was measured after 48 h using ELISA. Results for one representative experiment is shown.

FIGS. 14A-14C show that the inhibition of inflammatory cytokine secretion by BRC-OA is dose-dependent. TNF-□ (A), IL-1β (B), and IL-6 (C) secreted by LPS-stimulated THP-1 cells (a macrophage model) were measured after co-culturing of THP-1 with 50% or 25% BRC-OA derived tissue extracts by ELISA. BRC-OA extracts from cadaveric donors (grey bars) show similar magnitudes of anti-inflammatory activity as BRC-OA extracts from lipoaspirate donors (blue bars). Results for one representative experiment is shown.

FIG. 15 shows that delipidization of BRC-OA results in a significant decrease of anti-inflammatory activity in comparison to BRC-OA anti-inflammatory activity. Anti-inflammatory activity was measured by inhibition of TNF-□, IL-1β, and IL-6 secretion from LPS-stimulated THP-1 cells after co-culturing with BRC-OA or delipidized BRC-OA. Bars are: mean+/−SD for 3 donors.

FIG. 16 shows that BRC-OA extracts in a dose-dependent manner inhibit enzymatic activity of MMP-13 protease. Activity of MMP13 was measured by cleavage of a chromogenic substrate over time in the presence of 50%, 25%, 12.5% BRC-OA extracts. MMP13+substrate without the BRC-OA extract served as a control (circles). OD values for BRC-OA extracts without MMP13 were subtracted from OD values for MMP13+BRC-OA extracts. One representative experiment is shown.

FIG. 17 shows that BRC-OA inhibits IL-1 induced secretion of MMP-13 by the SW1353 cell line, a chondrocyte model. MMP-13 secreted by IL-1-stimulated SW1353 cells in culture medium was measured after co-culturing of SW1353 with BRC-OA using ELISA. Results for 3 BRC-OA donors are shown.

FIG. 18 shows that BRC-OA protects SW1353 cells from ROS-induced cell death. SW1353 cells were incubated with 300 □M H2O2 in the presence of BRC-OA for 24 h. ATP in viable cells was used to measure cell viability. Cell viability results are expressed in % of cell viability in experimental wells versus % of cell viability in control wells when SW1353 cells were incubated in medium only (grey bar). Cell viability in the control wells was considered 100%. BRC-OA derived from 5 different donors significantly increased cell survival in the presence of H2O2: 56% average cell viability in the presence of BRC-OA vs <10% in wells with H2O2 only (red bar).

FIG. 19 shows levels of 6 key anti-inflammatory, anti-oxidant, and protease inhibiting molecules in BRC-OA. Bars are: mean+/−SD.

FIG. 20 shows that that BRC-OA inhibits IL-1□ significantly stronger than the unprocessed adipose tissue. LPS-stimulated THP-1 cells (a monocyte/macrophage model) were co-cultured with 0.1g/well of the BRC-OA or unprocessed adipose tissue. IL-1□ secreted by LPS-stimulated THP-1 cells was measured after 48 h using ELISA. Bars are mean+/−SD for 3 donors.

FIG. 21 shows that on day 3 after SC implantation in rats, hBRC001 (Comprised of BRC-OA+SVF cells) and hBRC-OA became populated by rat M2 macrophages (CD206 marker with the molecular weight of ˜220 kilodalton (kDa)). M2 macrophages were detectable for 14 and 10 days in hBRC001 and hBRC-OA, respectively (the upper panel). Rat IL-10, a 17 kDa anti-inflammatory cytokine secreted by M2 macrophages, was detected in both hBRC001and hBRC-OA, and its levels increased overtime (the middle panel). There were no detectable CD206-positive cells and rat IL-10 in the adipose formulation on day 0 (prior to implantation). The lower panel shows Ponceau S-stained membrane confirming an equal protein load for all samples.

FIG. 22 shows IHC staining of hBRC-OA for CD206, a marker of M2 macrophages. Results show that as early as on day 3 after SC implantation in rats, hBRC-OA became populated by rat M2 macrophages (red arrows). There were no detectable CD206-positive cells in hBRC-OA formulation on day 0 (prior to implantation).

FIG. 23 shows that BRC-OA extract induces MSC migration (Right image). Left image—MSC migration in DMEM without FBS (negative/background control). Middle image—MSC migration in DMEM with 10% FBS (positive control). Images from one representative experiment are shown.

FIG. 24 shows microscopic appearance of cells that were isolated from hBRC001 and hBRC-OA 7 days after SC implantation in rats.

FIG. 25 shows a schematic description of a rat model of chemically induced osteoarthritis. BRC-OA safety and therapeutic effects are under evaluation after intra-articular administration in a 42-day mono-iodoacetate (MIA) induced knee pain model in immune compromised rats.

FIG. 26 shows absolute force threshold (eVF) for each animal group. Mean+/−SE, N=6/group, except N=3 for the non-diseased group.

FIG. 27 shows hindlimb dynamic weight bearing (DWB) percentages for each animal group. Mean+/−SE, N=6/group, except N=3 for the non-diseased group.

FIG. 28 shows histopathologic scores of synovial inflammation, synovial fibrosis, cartilage damage/loss, bone/calcified cartilage resorption, and osteophytes for each animal group. Mean+/−SE, N=6/group, except N=3 for the non-diseased group.

FIGS. 29A-29B shows MCP-1 levels in blood serum (A) and knee lavage (B). Bars are mean+/−SD for N=3 for the non-diseased group (Control—no OA) and N=6 for all other groups for blood serum (A) or N=3 for knee lavage.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed, it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an adipose tissue” includes a plurality of such adipose tissues, reference to “the adipose tissue” is a reference to one or more adipose tissues and equivalents thereof known to those skilled in the art, and so forth.

The word “or” as used herein means any one member of a particular list and also includes any combination of members of that list.

As used herein, the term “subject” or “patient” can be used interchangeably and refer to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

By “treat” is meant to administer one or more of the disclosed compositions (e.g. a composition comprising an adipose matrix comprising devitalized and partially delipidized adipose tissue) to a subject, such as a human or other mammal (for example, an animal model) that has or has an increased susceptibility for developing osteoarthritis, in order to prevent or delay a worsening of the effects of the disease or condition, or to partially or fully reverse the effects of the disease or condition (e.g., osteoarthritis). For example, “treat” can mean to prevent disease progression.

By “prevent” is meant to minimize the chance that a subject who has an increased susceptibility for developing a disease or disorder will develop the disease or disorder such as osteoarthritis.

As used herein, the terms “administering” and “administration” refer to any method of providing an adipose matrix or composition (e.g., a composition comprising an adipose matrix comprising devitalized and partially delipidized adipose tissue) as described herein, to a subject. Such methods are well known to those skilled in the art and include, but are not limited to: intra-articular administration, subcutaneous administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and oral administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject.

An “effective amount” of a composition as provided herein is meant a sufficient amount of one or more of the disclosed compositions (e.g., a composition comprising an adipose matrix comprising devitalized and partially delipidized adipose tissue) to provide the desired effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “effective amount.” However, an appropriate “effective amount” may be determined by one of ordinary skill in the art using only routine experimentation. The term “therapeutically effective amount” means an amount of a therapeutic, prophylactic, and/or diagnostic agent (e.g., adipose matrix) that is sufficient, when administered to a subject suffering from or susceptible to a specific disease or condition (e.g., osteoarthritis) to treat, alleviate, ameliorate, relieve, alleviate symptoms of, prevent, delay onset of, inhibit progression of, reduce severity of, and/or reduce incidence of the disease or condition.

“Natural”, in the context of, for example, “natural adipose tissue,” refers to properties exhibited by the adipose tissue in its native state in the subject or donor.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers, or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers, or steps that are not listed in the step.

### B. Compositions

As described herein, a composition can be used to refer to the adipose matrix alone or the adipose matrix in combination with something else, such as MSCs or a pharmaceutically acceptable carrier.

Disclosed are compositions comprising an adipose matrix. Disclosed are adipose matrices comprising devitalized and partially delipidized adipose tissue. Thus, in some aspects the disclosed composition is the tissue component of BRC001. In some aspects, the adipose tissue can be white adipose or brown adipose. Disclosed are compositions comprising any of the disclosed adipose matrices. For example, disclosed are compositions comprising an adipose matrix comprising devitalized and partially delipidized adipose tissue.

In some aspects, the term “devitalized” means at some point 100% of the native cells (e.g., cells that originated from the adipose tissue) have been killed and at least a portion of the killed cells (i.e., nonviable cells) are present in the adipose tissue. In some aspects, the term “devitalized” means 95, 96, 97, 98, 99, or 100% of the native cells (e.g., cells that originated from the adipose tissue) have been killed and at least a portion of the killed cells (i.e., nonviable cells) are present in the adipose tissue. In some aspects, the term “devitalized” means 75, 80, 85, 90, or 95% of the native cells (e.g., cells that originated from the adipose tissue) have been killed. In some aspects, a devitalized adipose tissue can later be populated with cells; however, if the adipose tissue was initially devitalized then even after populating with cells at a later step the adipose tissue can still be referred to as devitalized. In some aspects, devitalized adipose tissue can mean that some of the dead cells can be lost during processing but at least 98% of the dead cells are still present in the tissue.

In some aspects, the partially delipidized adipose tissue comprises at least 50% of the original lipids. In some aspects, the partially delipidized adipose tissue comprises at least 20% of the original lipids. In some aspects, the partially delipidized adipose tissue comprises at least 10%, 15%, 20%, 25%, 30, 35, 40%, 45%, 50%, 55%, 60%, 65%, 70%, 75%, 80%, 85%, or 90% of the original lipids. In some aspects, the partially delipidized adipose tissue comprises less than 100% of the original lipids but more than 5% of the original lipids. Thus, in some aspects, a partially delipidized adipose tissue does not have 100% of the original lipids of the adipose tissue but also is not completely devoid of lipids.

In some aspects, the devitalized and partially delipidized adipose tissue can be minced or micronized. In some aspects, the minced adipose tissue comprises pieces of adipose tissue 1 mm or less in size. In some aspects, devitalized and partially delipidized adipose tissue can be sieve minced via 2, 1 and 0.71 mm sieves. In some aspects, larger pieces can pass through the sieve, however, they can have a diameter of 1 mm or less. In some aspects, the minced or micronized adipose tissue is a homogenous population of pieces of adipose tissue less than 1 mm in size. In some aspects, the minced or micronized adipose tissue is a non-homogenous population of pieces of adipose tissue less than 1 mm in size.

In some aspects, the adipose matrix comprises about 0.1-20 μg DNA per mg of adipose matrix.

In some aspects, the adipose tissue (i.e. the partially delipidized adipose tissue) comprises all of the components of native adipose tissue except for a portion of the lipids and some cells. In some aspects, resident adipose tissue cells, such as Adipocytes, MSCs and some macrophages, remain in the tissue. In some aspects, the native adipose tissue factors that can provide therapeutic effects include, but are not limited to, adiponectin, leptin, vascular endothelial growth factors (VEGFs), platelet-derived growth factors (PDGFs), fibroblast growth factors (FGFs), IL-6, IL-8, insulin like growth factors (IGFs), and hepatocyte growth factor (HGF).

In some aspects, the adipose matrix is derived from the adipose of the subject who is being administered the composition. In some aspects, the adipose matrix is derived from the adipose of a subject different from the subject who is being administered the composition. In some aspects, the adipose matrix is derived from a cadaver. In some aspects, the adipose matrix is derived from a lipoaspirate.

In some aspects, the compositions further comprise mesenchymal stem cells (MSCs). For example, disclosed are compositions comprising an adipose matrix comprising devitalized and partially delipidized adipose tissue and MSCs. In some aspects, the MSCs can be differentiated, undifferentiated, or a combination thereof. In some aspects, the MSCs are adipogenic differentiated MSCs. In some aspects, the MSCs are a combination of undifferentiated MSCs and adipogenic differentiated MSCs. In some aspects, the MSCs have been cultured prior to combining with the adipose matrix.

In some aspects, the MSCs are allogeneic to the adipose tissue. For example, the adipose tissue can be derived from a first subject and the MSCs derived from a second subject, wherein the first and second subjects are not the same subject. In some aspects, the MSCs are autologous to the adipose tissue. For example, the adipose tissue and the MSCs can be derived from the same subject. In some aspects, MSCs can be isolated (and in some instances culturally expanded) from an adipose tissue and then after devitalizing and delipidizing the adipose tissue the MSCs can be added back to the tissue. Thus, the MSCs can be autologous. In some aspects, the MSCs can be from two or more subjects. In some aspects, the MSCs can be a combination of both autologous and allogeneic MSCs.

In some aspects, the cells can be originally derived from the adipose tissue but still exogenously added to the adipose matrix. For example, MSCs can be removed from the adipose tissue, then the adipose tissue can be devitalized and/or partially delipidized and then the MSCs can be added back to the adipose matrix. In this scenario, the MSCs are native to the adipose matrix as they are the tissues own naturally derived cells, but they are still exogenously added. In some aspects, the adipose matrix has no native cells present. Adipose matrix that has no native cells present means there are no cells that originated in the adipose tissue still present. Thus, any viable cells present in the adipose matrix would be from a different tissue or subject.

In some aspects, the MSCs are present in an amount of 5×10⁵ to 50×10⁶ MSCs per 1 milliliter of devitalized and partially delipidized adipose tissue.

In some aspects, the composition or adipose tissue can be, has previously been, or is cryopreserved or lyophilized. In some aspects, the composition or adipose tissue is not cryopreserved or lyophilized. In some aspects, the composition or adipose tissue can be considered “wet” in that it is not dried (e.g. lyophilized, air or vacuum-dried) but rather is stored in a solution. For example, wet adipose tissue can be stored in a cryopreservation solution, but just not cryopreserved (e.g., stored at 0° C. and higher temperatures). In some aspects, the wet composition or adipose tissue can be stored at room temperature or 4° C. for up to 3 months, 6 months, or 1 year and retain therapeutic properties.

### 1. Pharmaceutical Compositions

Disclosed are pharmaceutical compositions comprising any of the adipose matrices or compositions disclosed herein and a pharmaceutically acceptable carrier. For example, disclosed are pharmaceutical compositions comprising a composition comprising an adipose matrix comprising devitalized and partially delipidized adipose tissue and a pharmaceutically acceptable carrier.

Disclosed herein are pharmaceutical compositions that comprise one or more of the compositions disclosed herein. In an aspect, the pharmaceutical composition can comprise an adipose matrix comprising devitalized and partially delipidized adipose tissue. In an aspect, the pharmaceutical composition can comprise an adipose matrix comprising devitalized and partially delipidized adipose tissue and MSCs.

In some aspects, the pharmaceutical compositions can further comprise a pharmaceutically acceptable carrier. In some aspects, the pharmaceutical compositions described herein can be sterile and contain any of the disclosed compositions for producing the desired response in a unit of weight or volume suitable for administration to a subject. In some aspects, the pharmaceutical compositions can contain suitable buffering agents, including, e.g., acetic acid in a salt; citric acid in a salt; boric acid in a salt; and phosphoric acid in a salt.

When administered, the disclosed compositions or pharmaceutical compositions can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the disclosed compositions, and with each other, in a manner such that there is no interaction that would substantially impair the desired pharmaceutical efficacy.

As used herein, the term “pharmaceutically acceptable carrier” refers to solvents, dispersion media, coatings, antibacterial, isotonic and absorption delaying agents, buffers, excipients, binders, lubricants, gels, and surfactants that can be used as media for a pharmaceutically acceptable substance. The pharmaceutically acceptable carriers can be lipid-based or a polymer-based colloid. Examples of colloids include liposomes, hydrogels, microparticles, nanoparticles, and micelles. The compositions can be formulated for administration by any of a variety of routes of administration, and can include one or more physiologically acceptable excipients, which can vary depending on the route of administration. Any of the compositions described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions disclosed herein can be sterile and sterilized by conventional sterilization techniques or sterile filtered. Aqueous solutions can be packaged for use as is, or lyophilized; the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions, and sterile packaged powders. The active ingredient can be any of the growth hormone releasing hormone peptides described herein in combination with one or more pharmaceutically acceptable carriers. As used herein “pharmaceutically acceptable” means molecules and compositions that do not produce or lead to an untoward reaction (i.e., adverse, negative, or allergic reaction) when administered to a subject as intended (i.e., as appropriate).

In some aspects, administration of disclosed compositions or pharmaceutical compositions disclosed herein can be administered to mammals other than humans, e.g., for testing purposes or veterinary therapeutic purposes, and can be carried out under substantially the same conditions as described above.

### 2. Cryopreserved or Lyophilized Compositions

In some aspects, the disclosed compositions or adipose matrices can be cryopreserved. In some aspects, the disclosed compositions or adipose matrices can further comprise a cryopreservation solution. In some aspects, the disclosed compositions or adipose matrices can be previously cryopreserved. In some aspects, the disclosed compositions or pharmaceutical compositions can be cryopreserved. In some aspects, the disclosed compositions or pharmaceutical compositions can further comprise a cryopreservation solution. In some aspects, the disclosed compositions or pharmaceutical compositions can be previously cryopreserved. In some aspects, the disclosed MSCs can be cryopreserved. In some aspects, the disclosed MSCs can further comprise a cryopreservation solution. In some aspects, the disclosed MSCs can be previously cryopreserved. “Previously cryopreserved” can mean a composition that was once cryopreserved but has been removed (e.g., thawed) from the cryopreserved state.

Thus, for example, disclosed are compositions comprising an adipose matrix comprising devitalized and partially delipidized adipose tissue and further comprising a cryopreservation solution. A further example includes, compositions comprising an adipose matrix comprising devitalized and partially delipidized adipose tissue and MSCs, and further comprising a cryopreservation solution.

Disclosed are cryopreserved adipose matrices and compositions prepared using the disclosed methods of micronizing adipose tissue; emulsifying the micronized adipose tissue; centrifuging the micronized adipose tissue to produce at least two distinct layers, a top layer and a layer directly underneath the top layer, wherein free lipids are present in the top layer and a partially delipidized adipose tissue is present in the layer directly underneath the top layer; and removing the free lipids and freezing the adipose tissue before or after any of these steps to devitalize the adipose tissue. In some aspects, a third layer is present beneath the layer comprising the partially delipidized adipose tissue. In some aspects, the third layer comprises PBS from the wash.

Disclosed herein are compositions or adipose matrices comprising devitalized and partially delipidized adipose tissue. Disclosed herein are cryopreserved compositions comprising an adipose matrix comprising devitalized and partially delipidized adipose tissue. Disclosed herein are cryopreserved compositions comprising an adipose matrix comprising devitalized and partially delipidized adipose tissue and MSCs. In some aspects, both the adipose matrix and the MSCs are cryopreserved in a single composition. In some aspects, the adipose matrix and the MSCs are cryopreserved in multiple compositions and later combined into a single composition.

In some aspects, the cryopreserved adipose matrices and/or compositions comprise at least 10% native lipids. In some aspects, the cryopreserved adipose matrix or composition, when thawed, can comprise at least 10% lipids. In some aspects, the cryopreserved or previously cryopreserved adipose matrices or composition can comprise greater than 10%, 15%, 20%, 25%, 30%, 35%, 40%, 45%, 50%, 55%, 60%, 65%, 70%, 75%, 80%, 85%, 90%, 95%, or 99% lipids. In some aspects, the cryopreserved or previously cryopreserved adipose matrix or composition can be cut to a desired size. The percent of lipids present after thawing is at least 10, 20, 30, 40, 50, 60, 70, 80, 90, or 100% of the lipids present immediately prior to cryopreservation (but after the partial delipidization step).

In some aspects, a cryopreservation solution can contain one or more non-cell permeating cryopreservatives. Examples of non-cell permeating cryopreservatives, include but not limited to, polyvinyl pyrrolidione, a hydroxyethyl starch, a polysaccharide, a monosaccharide, an alginate, trehalose, raffinose, dextran, human serum albumin, Ficoll, lipoproteins, polyvinyl pyrrolidone, hydroxyethyl starch, autologous plasma, or a mixture thereof. In some aspects, the cryopreservative does not contain DMSO or glycerol. Further, a cryopreservation solution can contain serum albumin or other suitable proteins to stabilize the disclosed compositions during the freeze-thaw process and to reduce the damage to cells, thereby maintaining viability. In some aspects, a cryopreservation solution can contain a physiological solution, such as a physiological buffer or saline, for example phosphate buffer saline. In some aspects, a cryopreservation solution can comprise a lyoprotectant, such as trehalose or trehalose in combination with one or more antioxidants.

In some aspects, the disclosed compositions or adipose matrices can be lyophilized.

Disclosed are lyophilized adipose matrices comprising devitalized and partially delipidized adipose tissue. Disclosed are lyophilized compositions comprising an adipose matrix comprising devitalized and partially delipidized adipose tissue. Also disclosed are lyophilized compositions comprising an adipose matrix comprising devitalized and partially delipidized adipose tissue and MSCs.

Disclosed are lyophilized adipose matrices and compositions prepared using the disclosed methods of micronizing adipose tissue; emulsifying the micronized adipose tissue; centrifuging the micronized adipose tissue to produce at least two distinct layers, a top layer and a layer directly underneath the top layer, wherein free lipids are present in the top layer and a partially delipidized adipose tissue is present in the layer directly underneath the top layer; and removing the free lipids, freezing the adipose tissue before or after any of these steps to devitalize the adipose tissue, and lyophilizing the partially delipidized and devitalized adipose matrix. In some aspects, a third layer is present beneath the layer comprising the partially delipidized adipose tissue. In some aspects, the third layer comprises PBS from the wash.

In some aspects, the disclosed lyophilized adipose matrices or compositions comprise less than 15% residual water. In some aspects, the disclosed lyophilized adipose matrices or compositions comprise 5-12% residual water. In some aspects, the disclosed lyophilized adipose matrices or compositions comprise ≤5% residual water.

In some aspects, the disclosed lyophilized adipose matrices or compositions comprise trehalose. In some aspects, the disclosed lyophilized adipose matrices or compositions comprise trehalose, wherein the trehalose is present at a concentration of 0.25M-1.5M.

In some aspects, the lyophilized composition, when reconstituted can comprise at least 70% viable MSCs compared to the amount of MSCs prior to lyophilizing. In some aspects, reconstituted tissue can comprise greater than 40%, 45%, 50%, 55%, 60%, 65%, 70%, 75%, 80%, 85%, 90%, 95%, or 99% of the MSCs present prior to lyophilization. In some aspects, after reconstituting the lyophilized tissue, the tissue can then be cut to a desired size.

In some aspects, the lyophilized adipose matrices or compositions disclosed herein can be stable for at least three weeks. In some aspects, the lyophilized adipose matrices or compositions can be stable for at least three months. In some aspects, the lyophilized compositions can be stable for 1, 2, 3, 4, 5, 6, 7, 8, 9, 10, 11, 12, 24, 36, 48, or 60 months.

In some aspects, the lyophilized compositions disclosed herein can be reconstituted resulting in a reconstituted tissue. The described lyophilized adipose matrices or compositions can be reconstituted using standard techniques known in the art. In some aspects, reconstituting refers to rehydrating. Thus, the disclosed lyophilized adipose matrices or compositions can be reconstituted or rehydrated using water, saline, a buffer such as, but not limited to phosphate buffered saline (PBS), in a solution comprising a stabilizing agent such as, but not limited to bovine serum albumin (BSA), Plasma-Lyte A, or other clinically available electrolyte solutions, with human bodily fluids or a combination thereof. For example, lyophilized adipose matrices or compositions can be applied directly to a wound or tissue injury on a subject, and the subject's bodily fluids can reconstitute. In some aspects, a combination of bodily fluids and another known rehydrating solution can be used. Also, disclosed are reconstituted adipose matrices or compositions prepared using the methods disclosed herein.

### C. Methods

### 1. Methods of Treating

Disclosed are methods of treating a subject having osteoarthritis comprising administering to the subject a composition, wherein the composition comprises an adipose matrix comprising devitalized and partially delipidized adipose tissue. In some aspects, the composition and/or adipose matrix can be any of those compositions or adipose matrices described herein.

Disclosed are methods of treating or ameliorating a symptom of osteoarthritis in a subject comprising: administering to the subject a composition, wherein the composition comprises an adipose matrix comprising devitalized and partially delipidized adipose tissue. In some aspects, the methods can inhibit inflammation and oxidative stress that will lead to reduction in pain and improve functionality and quality of life; thus, the symptoms being treated can be, but are not limited to, pain, functionality, and/or quality of life.

In some aspects of any of the disclosed methods, the composition can be administered intra-articularly. For example, intra-articularly can include administering in any joint such as, but not limited to, knees, hips, shoulders, wrists, ankles, hands, and fingers. In some aspects, the composition can be administered using any known technique for administering therapeutics to a subject. In some aspects, the composition can be administered using any of the routes of administration described herein.

In some aspects, the partially delipidized adipose tissue comprises at least 50% of the original lipids. In some aspects, the partially delipidized adipose tissue comprises at least 20% of the original lipids. In some aspects, the partially delipidized adipose tissue comprises at least 10%, 15%, 20%, 25%, 30%, 35%, 40%, 45%, 50%, 55%, 60%, 65%, 70%, 75%, 80%, 85%, or 90% of the original lipids. In some aspects, the partially delipidized adipose tissue comprises less than 100% of the original lipids but more than 5% of the original lipids. Thus, in some aspects, a partially delipidized adipose tissue does not have 100% of the original lipids of the adipose tissue but also is not completely devoid of lipids.

In some aspects, the devitalized and partially delipidized adipose tissue can be minced or micronized. In some aspects, the minced adipose tissue comprises pieces of adipose tissue 1 mm or less in size. In some aspects, devitalized and partially delipidized adipose tissue can be sieve minced via 2, 1 and 0.71 mm sieves. In some aspects, larger pieces can pass through the sieve, however, they can have a diameter of 1 mm or less. In some aspects, the minced or micronized adipose tissue is a homogenous population of pieces of adipose tissue less than 1 mm in size. In some aspects, the minced or micronized adipose tissue is a non-homogenous population of pieces of adipose tissue is 1 mm or less in size.

In some aspects, the adipose matrix comprises about 0.1-20 μg DNA per mg of adipose matrix.

In some aspects, the composition, or specifically the adipose matrix, comprises MSCs. The MSCs are adipogenic differentiated MSCs. In some aspects, the MSCs are allogeneic to the adipose matrix. In some aspects, the MSCs are autologous to the adipose matrix. In some aspects, the MSCs are present in an amount of 5×10⁵ to 50×10⁶ MSCs per 1 milliliter of devitalized and partially delipidized adipose tissue. In some aspects, the MSCs are culturally expanded. In some aspects, culturally expanded MSCs can be referred to as Stromal Vascular Fraction (SVFs)

In some aspects, the adipose matrix can be derived from the adipose of the subject who is being administered the composition (i.e., autologous adipose). In some aspects, the adipose matrix can be derived from the adipose of a subject different from the subject who is being administered the composition (i.e., heterologous adipose). In some aspects, the MSCs present in a composition comprising adipose matrix and MSCs can be from the subject who is being administered the composition. In some aspects, the MSCs present in a composition comprising adipose matrix and MSCs can be from one or more subjects who are different than the one being administered the composition. In some aspects, the MSCs present in a composition comprising adipose matrix and MSCs can be a combination of MSCs from the subject who is being administered the composition and from one or more subjects different from the one being administered the composition.

In some aspects, the adipose matrix can be derived from a cadaver and/or a lipoaspirate.

In some aspects, the composition or adipose matrix is cryopreserved or has been previously cryopreserved. In some aspects, the composition or adipose matrix is lyophilized or has been previously lyophilized. In some aspects, the composition or adipose matrix has not been cryopreserved and/or lyophilized. For example, in some aspects, the composition or adipose matrix can be considered “wet” as described herein. In some aspects, the term “wet” can mean that the adipose can contain a cryopreservative solution, which can serve as a preservative, but the adipose is not cryopreserved or lyophilized.

In some aspects, the MSCs, whether the MSCs in the composition administered to the subject or MSCs from the subject that infiltrate the adipose matrix once administered to the subject, can allow for paracrine secretion of growth factors and cytokines and undergo differentiation into adipocytes, thus providing therapeutic effects. M2 macrophages are known in the art to have anti-inflammatory activities. Thus, in some aspects, the presence of adipose matrix with and without MSCs resulting in infiltration of the adipose matrix by macrophages that become polarized into M2 macrophages, decreasing inflammation and protecting joints, is the therapeutic effect of the disclosed methods.

In some aspects, devitalized adipose tissue comprises <1% viable cells. In some aspects, devitalized adipose tissue comprises <3, 2, or 1% viable cells. In some aspects, devitalized adipose tissue comprises <25, 20, 15, 10, 5 or 1% viable cells.

In some aspects, treating a subject having osteoarthritis by administering one of the disclosed compositions involves an inhibition or reduction in inflammatory cytokines. In some aspects, one or more of TNF-□, IL-1β, and IL-6.

In some aspects, treating a subject having osteoarthritis or treating or ameliorating a symptom of osteoarthritis in a subject by administering one of the disclosed compositions involves an inhibition or reduction in MMP-13 protease activity. In some aspects, the inhibition or reduction in MMP-13 protease activity is due to an inhibition of IL-1 induced secretion of MMP-13.

In some aspects, disclosed are methods of treating or ameliorating a symptom of osteoarthritis in a subject comprising administering to the subject a composition, wherein the composition comprises devitalized and partially delipidized adipose tissue, wherein the symptom of osteoarthritis is inflammation, pain, or poor quality of life. Thus, in some aspects, the disclosed methods can inhibit inflammation and oxidative stress that can lead to reduction in pain and improve functionality and quality of life.

### 2. Methods of Inhibiting Inflammatory Cytokines

Disclosed are methods of inhibiting inflammatory cytokines in a subject having osteoarthritis comprising administering to the subject a composition, wherein the composition comprises an adipose matrix comprising devitalized and partially delipidized adipose tissue. In some aspects, the composition and/or adipose matrix can be any of those compositions or adipose matrices described herein.

In some aspects, the methods of inhibiting inflammatory cytokines result in increased anti-inflammatory activity in the subject. In some aspects, an increase in anti-inflammatory activity can be determined by the presence of M2 macrophages and IL-10.

In some aspects, the inflammatory cytokines are IL-1β, TNF-α, and IL-6. Thus, disclosed are methods of inhibiting one or more of IL-1β, TNF-α, and IL-6 in a subject having osteoarthritis comprising administering to the subject a composition, wherein the composition comprises an adipose matrix comprising devitalized and partially delipidized adipose tissue.

In some aspects of any of the disclosed methods, the composition can be administered intra-articularly. For example, intra-articularly can include administering in any joint such as, but not limited to, knees, hips, shoulders, wrists, ankles, hands, and fingers. In some aspects, the composition can be administered using any known technique for administering therapeutics to a subject. In some aspects, the composition can be administered using any of the routes of administration described herein.

In some aspects, the partially delipidized adipose tissue comprises at least 50% of the original lipids. In some aspects, the partially delipidized adipose tissue comprises at least 20% of the original lipids. In some aspects, the partially delipidized adipose tissue comprises at least 10%, 15%, 20%, 25%, 30%, 35%, 40%, 45%, 50%, 55%, 60%, 65%, 70%, 75%, 80%, 85%, or 90% of the original lipids. In some aspects, the partially delipidized adipose tissue comprises less than 100% of the original lipids but more than 5% of the original lipids. Thus, in some aspects, a partially delipidized adipose tissue does not have 100% of the original lipids of the adipose tissue but also is not completely devoid of lipids.

In some aspects, the devitalized and partially delipidized adipose tissue can be minced or micronized. In some aspects, the minced adipose tissue comprises pieces of adipose tissue is 1 mm or less in size. In some aspects, devitalized and partially delipidized adipose tissue can be sieve minced via 2, 1 and 0.71 mm sieves. In some aspects, larger pieces can pass through the sieve, however, they can have a diameter of 1 mm or less. In some aspects, the minced or micronized adipose tissue is a homogenous population of pieces of adipose tissue less than 1 mm in size. In some aspects, the minced or micronized adipose tissue is a non-homogenous population of pieces of adipose tissue less than 1 mm in size.

In some aspects, the adipose matrix comprises about 0.1-20 μg DNA per mg of adipose matrix.

In some aspects, the adipose matrix comprises MSCs. The MSCs are adipogenic differentiated MSCs. In some aspects, the MSCs are allogeneic to the adipose matrix. In some aspects, the MSCs are autologous to the adipose matrix. In some aspects, the MSCs are present in an amount of 5×10⁵ to 50×10⁶ MSCs per 1 milliliter of devitalized and partially delipidized adipose tissue. In some aspects, the MSCs are culturally expanded.

In some aspects, the adipose matrix can be derived from the adipose of the subject who is being administered the composition. In some aspects, the adipose matrix can be derived from the adipose of a subject different from the subject who is being administered the composition. In some aspects, the MSCs present in a composition comprising adipose matrix and MSCs can be from the subject who is being administered the composition. In some aspects, the MSCs present in a composition comprising adipose matrix and MSCs can be from one or more subjects who are different than the one being administered the composition. In some aspects, the MSCs present in a composition comprising adipose matrix and MSCs can be a combination of MSCs from the subject who is being administered the composition and from one or more subjects different from the one being administered the composition.

In some aspects, the adipose matrix can be derived from a cadaver and/or a lipoaspirate.

In some aspects, the composition or adipose matrix is cryopreserved or has been previously cryopreserved. In some aspects, the composition or adipose matrix is lyophilized or has been previously lyophilized. In some aspects, the composition or adipose matrix has not been cryopreserved and/or lyophilized. For example, in some aspects, the composition or adipose matrix can be considered “wet” as described herein. In some aspects, the term “wet” can mean that the adipose can contain a cryopreservative solution, which can serve as a preservative, but the adipose is not cryopreserved or lyophilized.

In some aspects, the MSCs, whether the MSCs in the composition administered to the subject or MSCs from the subject that infiltrate the adipose matrix once administered to the subject, can allow for paracrine secretion of growth factors and cytokines and differentiation into adipocytes, thus providing therapeutic effects. M2 macrophages are known in the art to have protective effects. Thus, in some aspects, the presence of MSCs, resulting in newly formed adipocytes, and M2 macrophages, decreasing inflammation and protecting joints, is the therapeutic effect of the disclosed methods.

In some aspects, devitalized adipose tissue comprises <1% viable cells. In some aspects, devitalized adipose tissue comprises <3, 2, or 1% viable cells.

### 3. Methods of Inhibiting MMP-13

Disclosed are methods of inhibiting MMP-13 in a subject having osteoarthritis comprising administering to the subject a composition, wherein the composition comprises an adipose matrix comprising devitalized and partially delipidized adipose tissue. In some aspects, the composition and/or adipose matrix can be any of those compositions or adipose matrices described herein.

In some aspects, inhibiting MMP-13 comprises inhibiting MMP-13 enzymatic activity and/or inhibiting secretion of MMP-13 by chondrocytes.

In some aspects of any of the disclosed methods, the composition can be administered intra-articularly. For example, intra-articularly can include administering in any joint such as, but not limited to, knees, hips, shoulders, wrists, ankles, hands, and fingers. In some aspects, the composition can be administered using any known technique for administering therapeutics to a subject. In some aspects, the composition can be administered using any of the routes of administration described herein.

In some aspects, the partially delipidized adipose tissue comprises at least 50% of the original lipids. In some aspects, the partially delipidized adipose tissue comprises at least 20% of the original lipids. In some aspects, the partially delipidized adipose tissue comprises at least 10%, 15%, 20%, 25%, 30%, 35%, 40%, 45%, 50%, 55%, 60%, 65%, 70%, 75%, 80%, 85%, or 90% of the original lipids. In some aspects, the partially delipidized adipose tissue comprises less than 100% of the original lipids but more than 5% of the original lipids. Thus, in some aspects, a partially delipidized adipose tissue does not have 100% of the original lipids of the adipose tissue but also is not completely devoid of lipids.

In some aspects, the devitalized and partially delipidized adipose tissue can be minced or micronized. In some aspects, the minced adipose tissue comprises pieces of adipose tissue 1 mm or less in size. In some aspects, devitalized and partially delipidized adipose tissue can be sieve minced via 2, 1 and 0.71 mm sieves. In some aspects, larger pieces can pass through the sieve, however, they can have a diameter of 1 mm or less. In some aspects, the minced or micronized adipose tissue is a homogenous population of pieces of adipose tissue less than 1 mm in size. In some aspects, the minced or micronized adipose tissue is a non-homogenous population of pieces of adipose tissue less than 1 mm in size.

In some aspects, the adipose matrix comprises about 0.1-20 μg DNA per mg of adipose matrix.

In some aspects, the adipose matrix comprises MSCs. the MSCs are adipogenic differentiated MSCs. In some aspects, the MSCs are allogeneic to the adipose matrix. In some aspects, the MSCs are autologous to the adipose matrix. In some aspects, the MSCs are present in an amount of 5×10⁵ to 50×10⁶ MSCs per 1 milliliter of devitalized and partially delipidized adipose tissue. In some aspects, the MSCs are culturally expanded.

In some aspects, the adipose matrix can be derived from the adipose of the subject who is being administered the composition. In some aspects, the adipose matrix can be derived from the adipose of a subject different from the subject who is being administered the composition. In some aspects, the MSCs present in a composition comprising adipose matrix and MSCs can be from the subject who is being administered the composition. In some aspects, the MSCs present in a composition comprising adipose matrix and MSCs can be from one or more subjects who are different than the one being administered the composition. In some aspects, the MSCs present in a composition comprising adipose matrix and MSCs can be a combination of MSCs from the subject who is being administered the composition and from one or more subjects different from the one being administered the composition.

In some aspects, the adipose matrix can be derived from a cadaver and/or a lipoaspirate.

In some aspects, the composition or adipose matrix is cryopreserved or has been previously cryopreserved. In some aspects, the composition or adipose matrix is lyophilized or has been previously lyophilized. In some aspects, the composition or adipose matrix has not been cryopreserved and/or lyophilized. For example, in some aspects, the composition or adipose matrix can be considered “wet” as described herein. In some aspects, the term “wet” can mean that the adipose can contain a cryopreservative solution, which can serve as a preservative, but the adipose is not cryopreserved or lyophilized.

In some aspects, the MSCs, whether the MSCs in the composition administered to the subject or MSCs from the subject that infiltrate the adipose matrix once administered to the subject, can allow for paracrine secretion of growth factors and cytokines and differentiation into adipocytes, thus providing therapeutic effects. M2 macrophages are known in the art to have protective effects. Thus, in some aspects, the presence of MSCs, resulting in newly formed adipocytes, and M2 macrophages, decreasing inflammation and protecting joints, is the therapeutic effect of the disclosed methods.

In some aspects, devitalized adipose tissue comprises <1% viable cells. In some aspects, devitalized adipose tissue comprises <3, 2, or 1% viable cells.

### 4. Delivery of Compositions

The disclosed adipose matrices and compositions comprising the adipose matrices can be delivered using a variety of well-known techniques.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder, or oily bases; and thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines, and substituted ethanolamines.

### EXAMPLES

### A. Example 1. Preparation of BRC001 Components and BRC001 Fabrication

### 1. General Statements:

Human adipose tissues were received from eligible adult living and cadaveric donors after obtaining written informed consent, and tissue regulations for receipt and disposition of tissues were strictly followed.

Animal studies were conducted in compliance with the current version of the following: 1) Animal Welfare Act Regulations (9 CFR); 2) U.S. Public Health Service Office of Laboratory Animal Welfare (OLAW) Policy on Humane Care and Use of Laboratory Animals; 3) Guide for the Care and Use of Laboratory Animals (Institute of Laboratory Animal Resources, Commission on Life Sciences, National Research Council, 1996); and 4) AAALACi accreditation. Procedures used in this study have been designed to avoid or minimize unacceptable discomfort, distress, or pain to the animals.

### 2. Preparation of Devitalized and Partially Delipidized Human Adipose Tissue (BRC001 Tissue Component=BRC-OA).

Adipose tissue was stored frozen at −80° C. prior to processing. Adipose tissue was cut using scissors into small (3-5 mm) pieces, and then with a “mezzaluna” knife until the tissue became a homogeneous mass with ˜1-2 mm tissue particle size. After that, tissue was filtered and washed with saline via 2 mm, 1 mm, and 0.71 mm sieves. Filtered and washed adipose was incubated at 37° C. overnight in a solution of antibiotics. On the next day, tissue was washed from antibiotics with a large volume of saline, and then centrifuged at room temperature at 400 g for 10 min. Free lipids from the top were removed with a pipette. Partially delipidized adipose was incubated in 0.5M trehalose-0.25% human serum albumin (HSA) preservative solution for 30 min, and then after decanting the preservative solution, tissue was aliquoted in cryovials and syringes. Packaged tissue was stored at −80° C. prior to use.

### 3. Preparation of Human Adipose-Derived SVF Cells (BRC001 Cell Component).

In contrast to the storage of adipose tissue frozen at −80° C. prior to processing of the BRC001 tissue component, preparation of BRC001 cells was always performed using fresh tissue. ˜5 mL of the fresh adipose tissue was cut with scissors into 3-5 mm pieces and digested in a 15 mL tube with 10 mL DMEM medium and 0.5 mL 2% collagenase type II solution. The tube was incubated on a rotator at 37° C. for 60 min. Digested tissue was filtered via a 0.3 □m nylon filter (by gravity). Cells were centrifuged 10 min at 400 g at RT. Top layers, containing free lipids and adipocytes, and the middle DMEM layer were removed. The cell pellet was resuspended in 15 mL DMEM-low serum, counted, and plated in 1×75 cm² flask for expansion. On the next day, the medium was changed allowing expansion of only cells attached to the tissue culture plastic. When cells reached 90% confluency, cells were collected from the flask using TrypLE enzyme and cryopreserved in CryoStor (10% DMSO) solution. Vials with cryopreserved cells were stored in liquid nitrogen or at −80° C. prior to use.

### 4. BRC001 Fabrication.

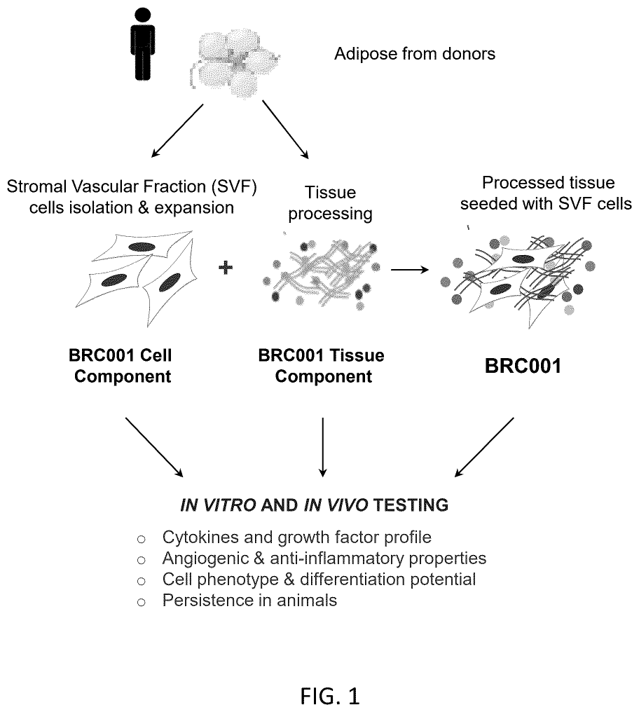

BRC001 cell component (culture expanded adipose derived SVF cells) were mixed with the BRC001 tissue component: 1-2 mil cells with 1 mL tissue. Formulated BRC001 was used for experiments on the day of preparation or cryopreserved in DMSO and trehalose containing preservation solution for storage prior to experiments. FIG. 1 shows a schematic of BRC001 processing, components, fabrication, and characterization.

### B. Example 2. BRC001 Cell and Tissue Components Characterization

### 1. BRC001 Cell Morphology and Phenotypes.

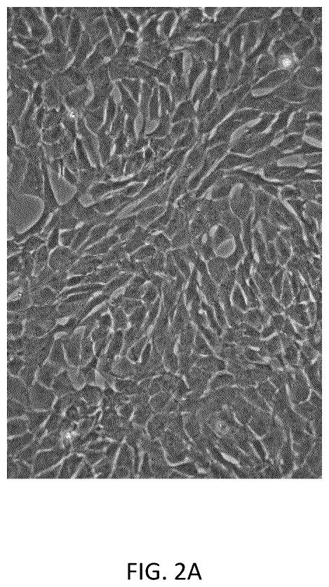

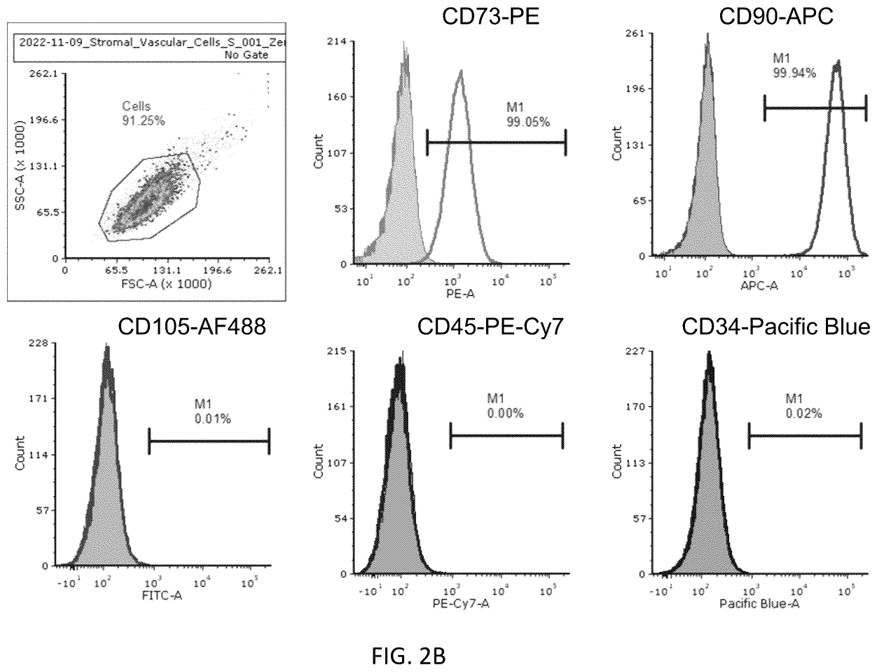

The cell morphology of SVF cells isolated from adipose and culture-expanded was evaluated microscopically, and phase contrast microscopic pictures were taken. As early as at P1, cells derived from 10 different donors showed a uniform adherent fibroblast-like cell morphology (FIG. 2A). Cell phenotype was evaluated by FACS using fluorescent-labeled antibodies CD73, CD90, CD105, CD45, and CD34 and isotype control antibodies. Cell staining was performed in a V-bottom 96-well plate. Cells were incubated with 5 mL antibody for 30 min at 4° C. The cells were washed 2 times with staining buffer. After washing, cells were transferred into 5 mL FACS tubes. Analysis was performed using BD FACSCanto clinical flow cytometry system. Representative results for one donor of cells are shown in FIG. 2B. These donors had >99% CD73 and CD90 positive cells and <0.02% CD45, CD34, and CD105 positive cells.

In conclusion, the cells showed a uniform phenotype: >96% positive (the lowest % among 10 tested donors) for mesenchymal stem cell (MSC) markers CD73 and CD90, and negative (<1%, the highest % among 10 tested donors) for CD45, CD34, and CD105 hematopoietic stem cell (HST) markers. Table 1 provides a list of FACS antibodies (all antibodies were from Biolegend, San Diego, CA).

TABLE 1
FACS antibodies

| | |
|---|---|
| 1 | CD45-PE-Cy7 |
| 2 | CD34-Pacific blue |
| 3 | CD105-Alexa488 |
| 4 | CD90-APC |
| 5 | CD73-PE |

### 2. BRC001 Cell Adipogenic Differentiation.

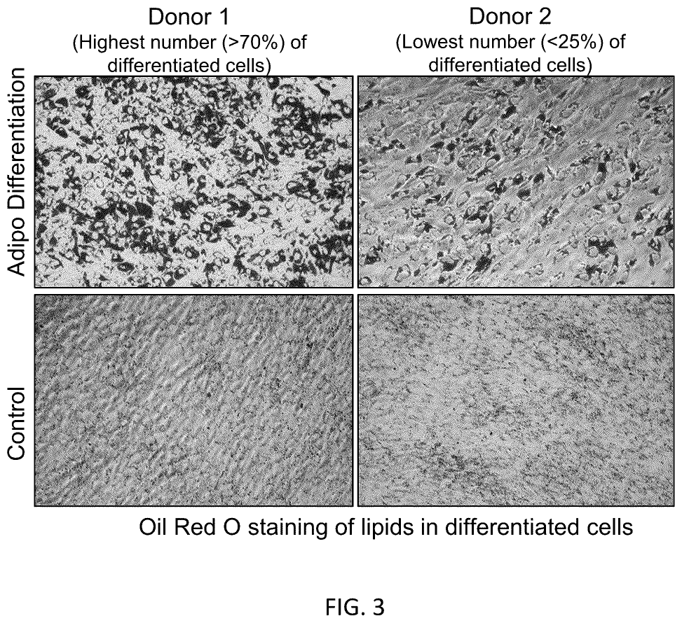

Culture expanded SVF cells were plated in wells of a 24-well plate. When cells formed a monolayer, DMEM-low serum culture medium was replaced by an adipogenic differentiation medium. Cells in DMEM-low serum served as a control. Medium was changed every 3-4 days. Cells were cultured for 7-10 days with microscopic assessment for the presence of cells with accumulated lipid vacuoles in the cytoplasm. After 7-10 days, cells in a 24-well plate were fixed in 4% formaldehyde and stained with Oil Red O according to a standard protocol. Photographs were taken. Adipogenic differentiation for each donor was graded semi-quantitatively based on the number of cells stained with Oil Red O. Cells from all tested donors had adipo-differentiation potential; however, the number of differentiated cells varied between donors from 25% to 75% of differentiated cells in a microscopic field. Microphotographs of adipo-differentiated cells for two donors with the lowest and the highest differentiation potentials are shown in FIG. 3.

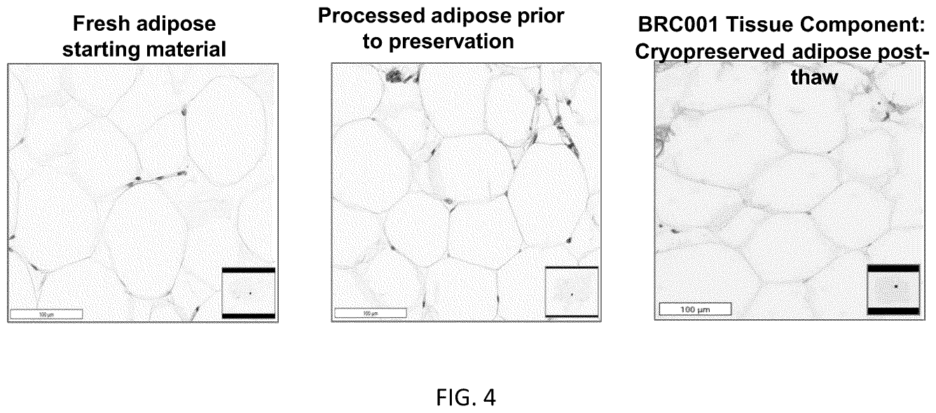

BRC001 tissue component structure. The structure of the BRC001 tissue component was evaluated histologically and compared to fresh adipose tissue prior to processing and to processed adipose tissue prior to preservation. Samples of fresh human adipose tissues prior to and after processing and cryopreserved post-thaw BRC001 tissue component were fixed in formalin for histological analysis. Fixed samples were stained by hematoxylin and eosin (H&E) according to the standard protocol at a certified histology lab. Stained samples were evaluated by an independent certified pathologist. Images of H&E-stained tissue sections are shown in FIG. 4. The structures of all samples are consistent with the structure of adipose tissue. There are no significant differences between samples. Thus, the structure of the native adipose is preserved in the BRC001 tissue component.

Cytokine and growth factor profile of the BRC001 cell and tissue components.

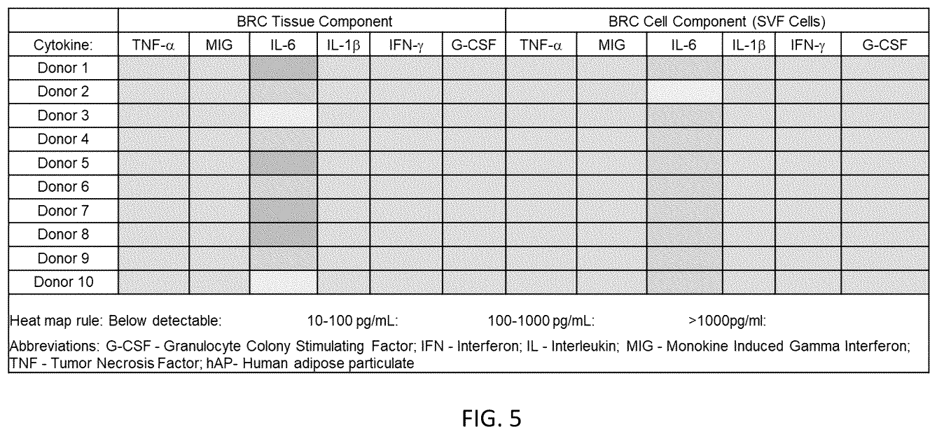

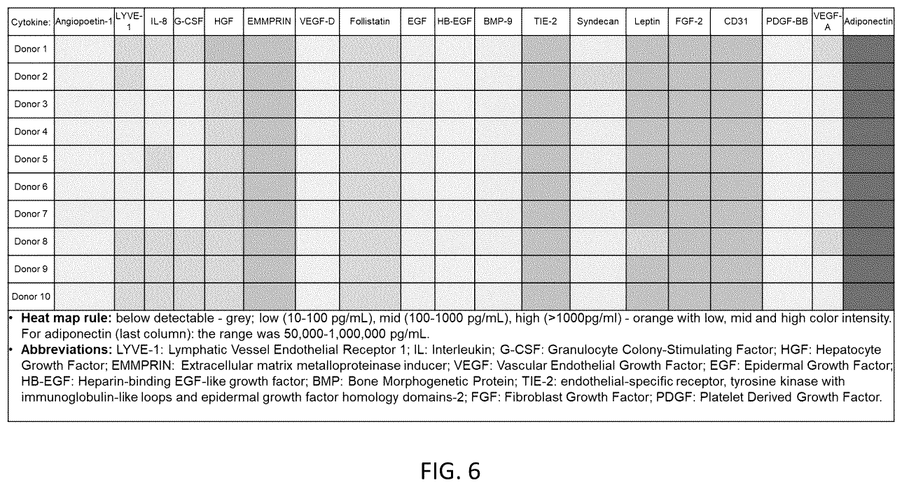

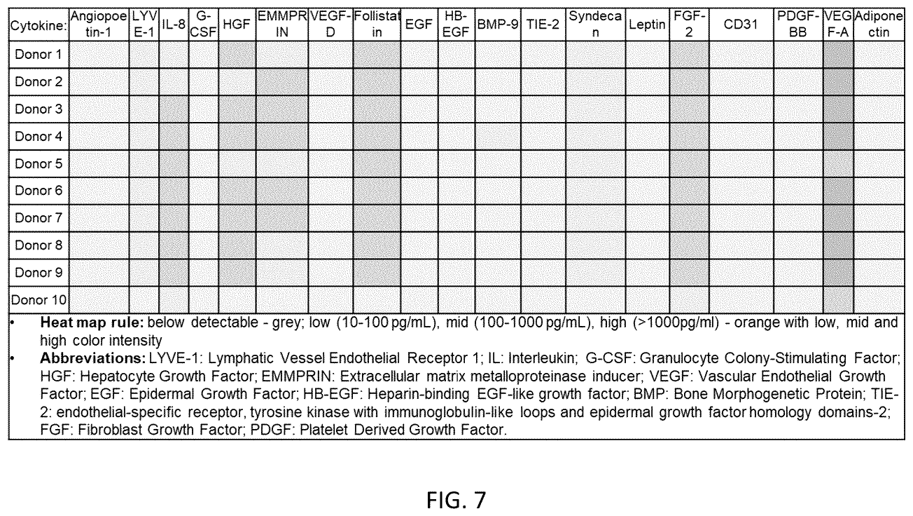

BRC001 tissue extract was prepared by mixing 1:1 (W/V) tissue and a medium in Eppendorf tubes. Tubes were vortexed and incubated on a rotator for 2 h at RT. After that, tubes were centrifuged in an Eppendorf centrifuge at RT for 10 min at 13,000 rpm. Tissue extracts were transferred into new Eppendorf tubes. All tubes were stored at −80° C. prior to testing. For the BRC001 cell component, a 72 h conditioned culture medium was collected from cultured cell monolayers in 24 well plates. Prepared BRC001 cell component conditioned medium and BRC001 tissue extracts were tested for cytokines and growth factors using multiplex Luminex assay kits (6-plex and 18-plex kits from ThermoFisher were used) and individual cytokine & growth factor ELISAs (VEGF, adiponectin) according to manufacturers' protocols. Results are summarized in FIGS. 5, 6 and 7. Results demonstrate that for the exception of IL-6, other inflammatory cytokines are at low or below detectable levels in the BRC001 cell and tissue components. Both components contain multifunctional growth factors (HGF, FGF2, and others); however, only the BRC001 cell component has high levels of angiogenic growth factor VEGF, and only the BRC001 tissue component contains adipokines (adiponectin, leptin).

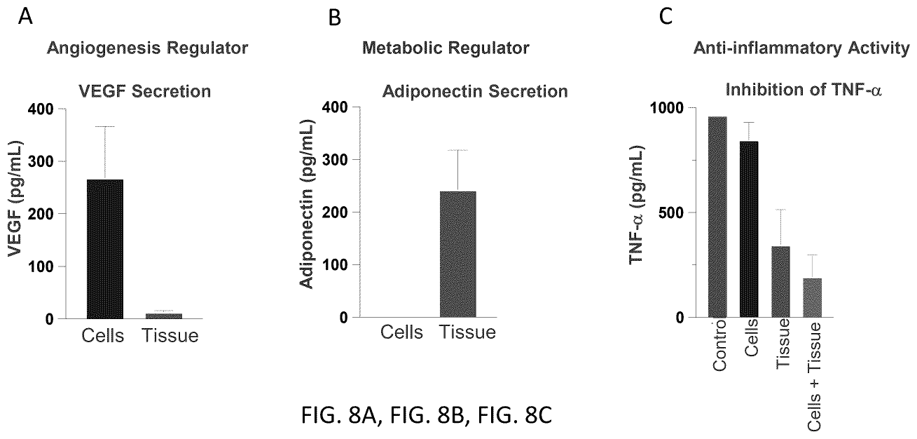

Anti-Anti-inflammatory activity of BRC001 and its components BRC001 cells and tissue. Anti-inflammatory activity of BRC001 and its components was evaluated using THP-1 cells, a model for human monocytes/macrophages. THP-1 cells (1 mil cells/mL/well of a 24-well plate) were stimulated by 0.5 □g/mL LPS in the presence of BRC001 or BRC001 tissue or BRC001 cells for 48 h, and then TNF-□ was measured in culture supernatants using ELISA. % inhibition of TNF-□ secretion in the presence of cells, tissue, and cells+tissue was calculated relative to THP-1+LPS control using a formula: % TNF-□ inhibition: 100−((TNF-□ in test sample/TNF-□ in THP-1+LPS)*100). Results of BRC001 and its components testing are shown in FIG. 8C. Although cells alone had some anti-inflammatory activity (20-35% of TNF-□ inhibition), results show that the BRC001 tissue component, but not cells, is the primary mediator of anti-inflammatory activity in BRC001. This was an unexpected finding because data in the literature indicate that SVF cells in adipose have anti-inflammatory activity. Moreover, BRC001 tissue, but not cells, is a reservoir of adipose-specific metabolic factors (i.e., adiponectin) (FIG. 8B). Anti-inflammatory activity and metabolic regulators are desirable for the treatment of osteoarthritis. BRC001 cell component is the major source of angiogenic factors like VEGF (FIG. 8A). VEGF is not necessarily desirable for the treatment of osteoarthritis, and one of the current approaches for a new OA therapy development is to inhibit VEGF in the OA knee. Thus, based on these unexpected findings and the known desirable and undesirable activities for the treatment of OA, the BRC001 tissue component alone is a good therapeutic candidate for OA treatment.

### C. Example 3. Persistence of BRC001 Components In Vivo

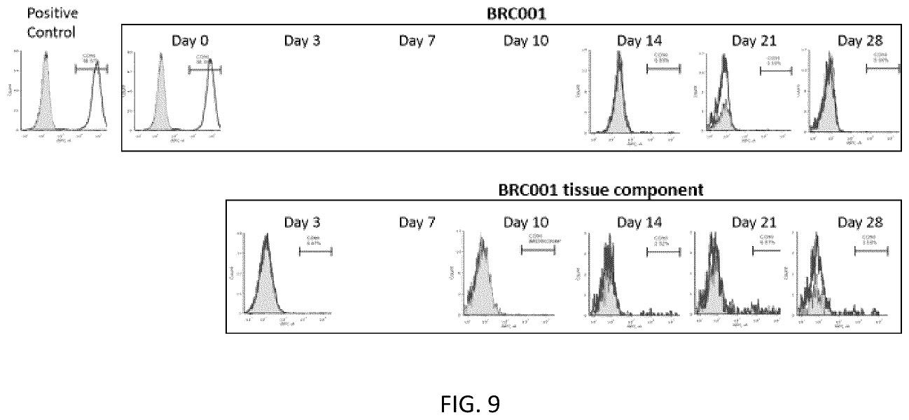

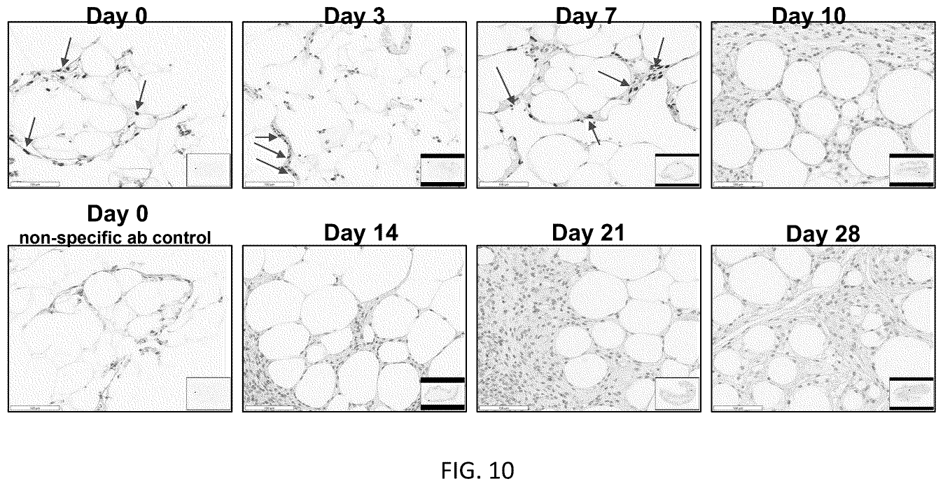

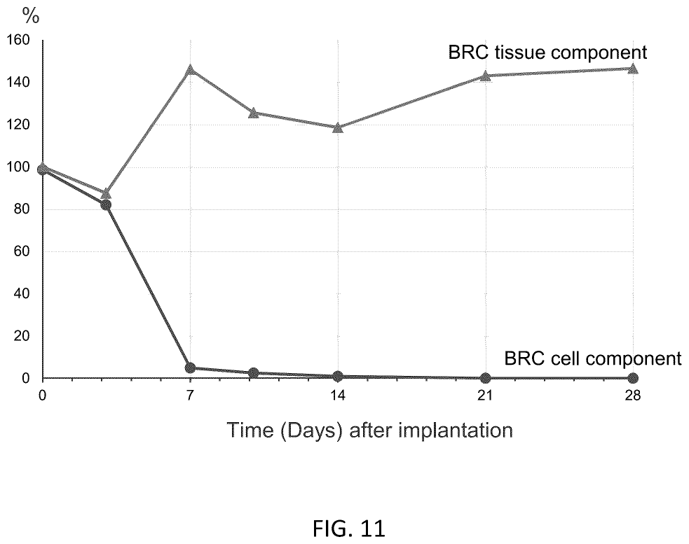

Persistence of BRC001 tissue and cells was evaluated for 4 weeks after subcutaneous implantation of BRC001 (tissue+cells) or BRC001 tissue alone in Sprague Dawley (SD) rats. The study was conducted using twelve 4-5 weeks old male SD rats. Animals were randomly divided in 2 groups: Group 1 (n=6), BRC001 (tissue+cells); and Group 2 (n=6), BRC001 tissue alone. All animals were weighed weekly for the study duration. Implantation of human adipose formulations was performed after one week acclimation (Day 0). All rats under general anesthesia received subcutaneously ˜0.5 mL/point of BRC001 (tissue+cells) (Group 1, n=6) or BRC001 tissue alone (Group 2, n=6) formulations in 4 points on the shaved dorsum (2 on the left and 2 on the right side of the spinal column, total 2 mL) using a 20G needle. Remaining formulations were fixed in 10% Neutral Buffer Formalin (NBF) for histological analysis. Euthanasia and necropsy sample collections from animals in Group 1 and Group 2 (one rat from each group) were performed on days 3, 7, 10, 14, 21, and 28 post-implantations. On euthanasia days, gross examination of animals was performed. The blood serum was collected from each animal. After terminal blood collection, the skin on the back of each rat was cut open, and the grafts were identified, photographed, dissected out, and weighed. One BRC001 graft from each animal was fixed in 10% NBF for histological analysis, another graft was collected in a tube containing medium with antibiotics for cell analysis, and the remaining two grafts were flash-frozen on dry ice for biochemical analyses. Sample analyses included: serum blood chemistry, histology of H&E (structure of the grafts) and immunohistochemistry of NUMA−1 (a human nuclear marker to assess cell persistence) stained adipose grafts, and FACS for human CD90-positive cells in the grafts. For FACS analysis, cells were isolated from the grafts by enzymatic digestion and stained with anti-human CD90-allophycocyanin (APC) or mouse IgG1-APC isotype control antibodies in FACS staining buffer (5 □L/well of a 96-V-bottom plate) for 30 min on wet ice. Cells were then washed twice with FACS staining buffer. Cells were transferred from the plate in 5 mL FACS tubes and analyzed using BD FACSCanto II. FACS and histologic analyses showed that human cells in BRC001 persisted in vivo for 7 days (FIGS. 9 and 10). FIG. 11 shows persistence of BRC001 tissue component (triangle dot line) and BRC001 cell component (circle line). Variability of the BRC001 tissue weight between time points is due to the presence of host tissues fused with the implants, and therefore it was not possible to dissect out the host tissue. % of weight for the tissue component and % of cell number for the cell component relative to the tissue weight and cell number on day 0 (prior to implantation) is shown in FIG. 11. Results show that the BRC001 tissue persisted for the entire duration of the study without decrease of graft weight (FIG. 11, triangle dot line); however, BRC001 cells persisted only for one week post-implantation (FIG. 11, circle dot line).

### D. Example 4. BRC-OA Characterization

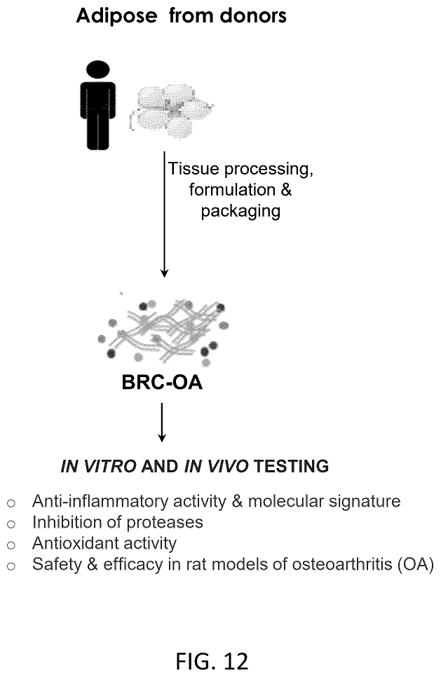

Experimental results show that the BRC001 tissue component alone without SVF cells has anti-inflammatory activity (FIG. 8C), contains metabolic regulators (FIG. 8B), and persists in vivo for a long period of time (FIGS. 9-11). These characteristics are desirable for OA treatment. At the present time, there are no off-the-shelf therapies on the market or in development that will have all these characteristics. Although autologous adipose has such properties, the treatment is not available off-the-shelf. This treatment requires harvesting and processing of adipose from a patient, which is time-consuming and may lead to adipose harvest site morbidity. Moreover, autologous adipose is not tested for potency prior to the use, and its properties can be altered by age and underlying diseases of the patient. These statements support development of BRC001 tissue component as a therapy for osteoarthritis. To be able to distinguish it from a tissue component of the BRC001 therapeutic formulation, it was renamed from BRC001 tissue component to BRC-OA. FIG. 12 shows a schematic of the BRC-OA formulation and its characterization.

### 1. Mechanisms of BRC-OA Anti-Inflammatory Activity.

Experiments described below were directed to answer several key questions regarding BRC-OA anti-inflammatory activity: Is BRC-OA anti-inflammatory activity mediated via direct contact between inflammatory cells and tissue? Does BRC-OA inhibit other inflammatory cytokines involved in the pathogenesis of OA? Can cadaveric donors be used as an adipose tissue source for BRC-OA? Is lipid retention in BRC-OA beneficial?

To answer these questions, the anti-inflammatory activities of BRC-OA tissue vs tissue-free extracts, BRC-OA derived from cadaveric vs lipoaspirate donors, and BRC-OA vs a delipidized version of BRC-OA were tested. Effects of BRC-OA on TNF-□, IL-1β and IL-6, three cytokines that are known to play a key role in development and progression of OA, were evaluated.

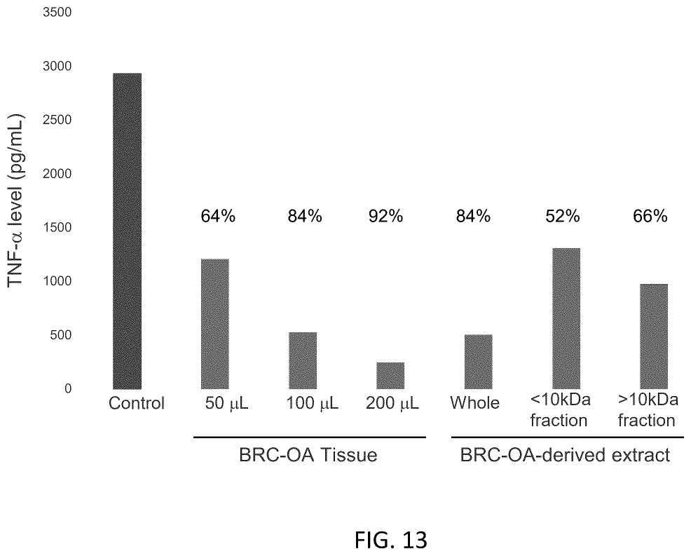

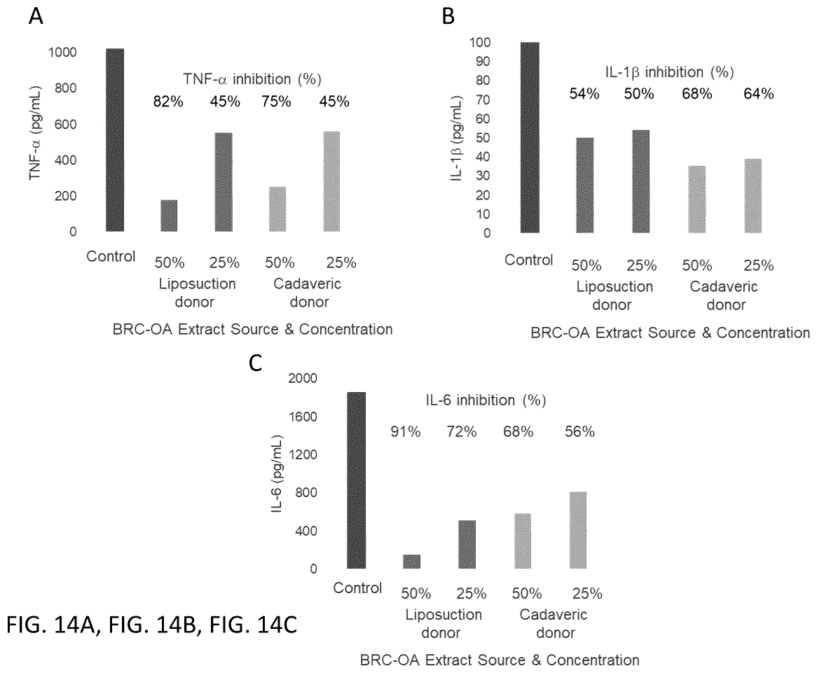

Anti-inflammatory activity of BRC-OA (tissue and extracts) was evaluated using THP-1 cells, a model for human monocytes/macrophages. THP-1 cells (1 mil cells/mL/well of a 24-well plate) were stimulated by 0.5 g/mL LPS in the presence of BRC-OA for 48 h, and then TNF-□ was measured in culture supernatants using ELISA. Tissue was tested at 50, 100, and 200 □L/well, and tissue extracts were tested at 25% and 50% concentrations. % inhibition of inflammatory cytokine secretion in the presence of BRC-OA was calculated relative to THP-1+LPS control using a formula: % inhibition: 100−((inflammatory cytokine in test sample/inflammatory cytokine in THP-1+LPS)*100). Results of BRC-OA testing are shown in FIGS. 13 and 14. Results show that BRC-OA inhibits TNF-□ secretion by LPS-stimulated THP-1 cells in a dose-dependent manner. The effect is mediated by soluble factors, and anti-inflammatory factors <10 kDa (most likely small molecules and peptides) and >10 kDa (most likely protein origin molecules) are present in BRC-OA. In addition to TNF-□, soluble factors in BRC-OA inhibit inflammatory cytokines IL-1β and IL-6 in a dose-dependent manner. BRC-OA derived from cadaveric adipose shows a similar magnitude of anti-inflammatory activity as BRC-OA derived from lipoaspirate donors (FIG. 14). Thus, adipose from cadaveric donors is an appropriate tissue source for BRC-OA: it retains anti-inflammatory activity, which is desirable for OA treatment.

### 2. Effects of Delipidization on Anti-Inflammatory Activity of BRC-OA.

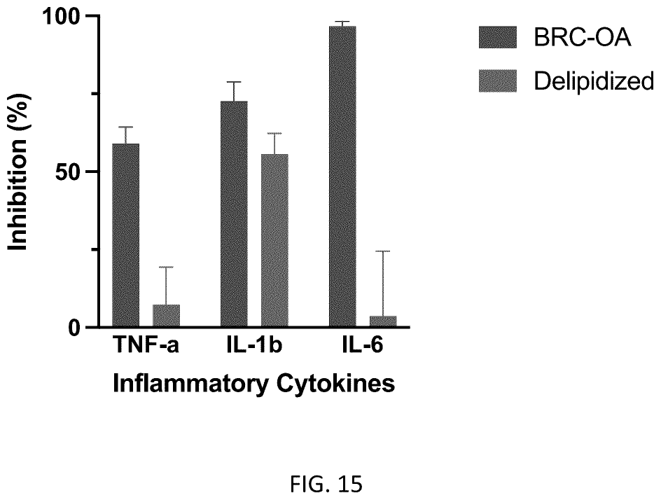

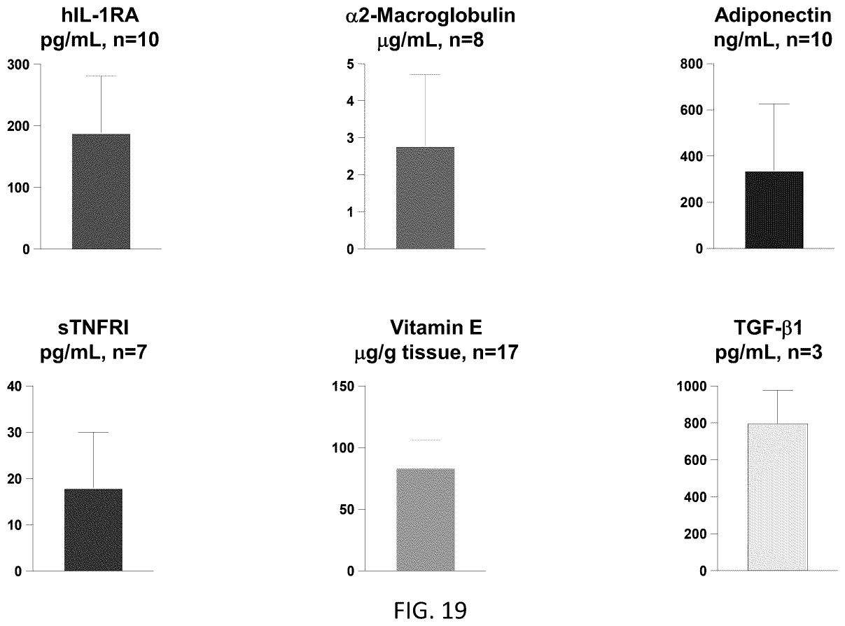

BRC-OA delipidization. 1 mL of BRC-OA from 3 donors was transferred into 10 mL syringes and emulsified in 5 mL sterile water by forcefully passing tissue between two connected syringes 50 times. After emulsification, tissues were transferred into 50 mL tubes. Tubes were filled with water to the 45 mL mark. Tubes were placed on a rotator for overnight incubation at 37° C. After overnight incubation, tubes were centrifuged for 20 min at RT at 1000 g to pellet the delipidized tissue. After centrifugation, the top layers of free oil, non-delipidized tissue, and water were decanted from tubes. Delipidized adipose was collected from 50 mL tubes and used for evaluation of anti-inflammatory activity at 0.1 mL/well concentration. The volumes of delipidized adipose was ˜0.1 mL, which is <10% of the starting BRC-OA volume. Anti-inflammatory activity of BRC-OA vs delipidized BRC-OA was evaluated using THP-1 cells as describe above. Results of the experiment are shown in FIG. 15. Results show that delipidization leads to significant decrease of anti-inflammatory activity (FIG. 15). Moreover, the concentration of delipidized BRC-OA used in the experiment corresponds to ˜1 mL of BRC-OA, which is 10-times higher than 0.1 mL/well BRC-OA used in the majority of experiments. Decrease of anti-inflammatory activity after delipidization can be explained by washing out most of the tissue resident anti-inflammatory factors. Moreover, lipids in adipose tissue are known to be the main reservoirs of fat-soluble vitamins A, D, and E. These vitamins have anti-inflammatory and anti-oxidant activities. BRC-OA contains a high level of vitamin E (FIG. 19). After delipidization, vitamin E was below the detectable level (data not shown).

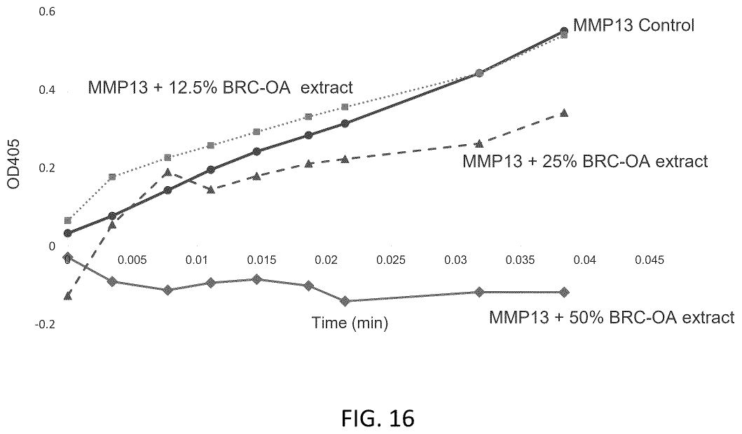

Inhibition of MMP-13 by BRC-OA. Matrix metalloproteinases (MMPs) are involved in cartilage degradation in OA. Particularly, MMP-13 has been identified as a key MMP involved in the cleavage of type II collagen, the main type of collagen in articular cartilage. Inhibition of MMP-13 is an attractive therapeutic target for OA. The potential of BRC-OA to inhibit MMP-13 enzymatic activity and secretion of MMP-13 by chondrocytes in an inflammatory microenvironment was investigated. Effects of BRC-OA on MMP-13 enzymatic activity was tested using an MMP-13 inhibitor screening kit (Abcam). Activity of MMP13 was measured by cleavage of a chromogenic substrate over time in the presence of 50%, 25%, and 12.5% BRC-OA extracts. MMP13+substrate without the BRC-OA extract served as a control. OD values for BRC-OA extracts without MMP13 were subtracted from OD values for MMP13+BRC-OA extracts. FIG. 16 shows results for one representative experiment. Results show that soluble factors in BRC-OA extract inhibit MMP13 activity in vitro in a dose-dependent manner: 50% extract—strong inhibition; 25% extract—weak inhibition; and 12.5% extract—no inhibition.

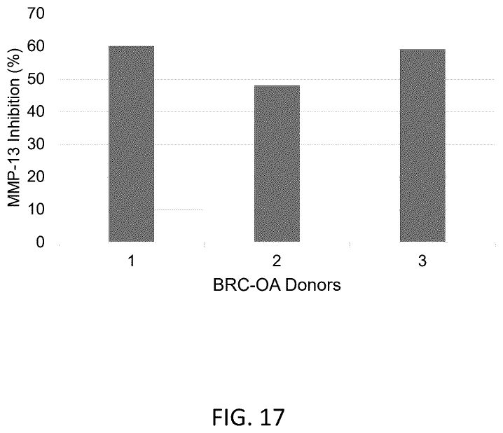

Evaluation of BRC-OA effects on IL-1□-induced MMP-13 secretion by SW1353 cells. SW1353 (ATCC) is a chondrosarcoma cell line that is often used as a chondrocyte model. Similar to primary chondrocytes, SW1353 cells significantly upregulate secretion of MMP-13 in the presence of IL-1□. SW1353 cells were seeded 50,000 cells/well in DMEM-10% FBS. After 24 h, the medium was removed, cells were washed with PBS, and 1 mL/well of fresh DMEM-10% FBS was added to each well. IL-1 (Proteintech) was used at a 10 ng/mL final concentration. BRC-OA was used at 0.1 mL/well. The plate was incubated for 24 h, and then culture supernatants from each well were collected. MMP-13 in culture supernatants was measured by ELISA (ProteinTech and R&D Systems). % inhibition of MMP-13 secretion in the presence of BRC-OA was calculated relative to SW1353+IL-1□ control using a formula: % inhibition: 100−((MMP-13 in test sample/MMP-13 in SW1353+IL-1□)*100). FIG. 17 shows that BRC-OA derived from 3 donors inhibits IL-1 induced secretion of MMP-13 by SW1353 cells by 50-60%.

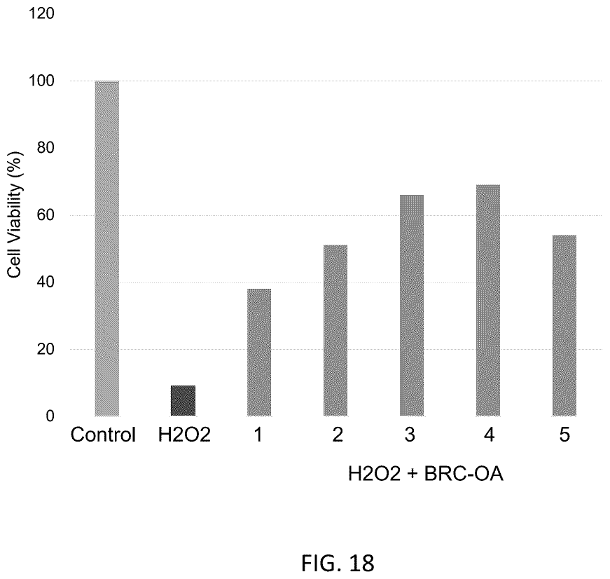

Anti-oxidant activity of BRC-OA: cell protection from ROS-induced cell death. Oxidative stress and reactive oxygen species (ROS) induced chondrocyte cell death is another hallmark of OA. Therefore, the protective effects of BRC-OA from ROS-induced cell death were evaluated. In these experiments, SW1353 cells were seeded 100,000 cells/well in DMEM-10% FBS. After 24 h, the medium was removed, cells were washed with PBS, and 1 mL/well of fresh DMEM-10% FBS was added to each well. H2O2 was added to designated wells at a 300 □M final concentration. BRC-OA was used at 0.1 mL/insert of a 24-well plate trans-wells. The plate was incubated for 24 h, trans-well inserts were then removed, and SW1353 cells in the plate were examined microscopically. Then, medium was removed from wells, and 0.25 mL of new medium were added. Cell viability was evaluated by ATP detection using the CellTiter-Glo 2 cell viability luminescent assay kit (Promega). 0.25 mL of the ATP reagent were added to each well of the 24-well plate containing 0.25 mL DMEM-10% FBS. After that, 0.2 mL/well was transferred from each well of the 24 well plate into 2 wells of a 96-F-bottom white plate. Luminescence reading of the plate was performed using the Victor plate reader. Cell viability was calculated in % cell viability in the control wells (SW1353 cells incubated in the medium only). Cell viability in the control wells was considered 100%. FIG. 18 shows that BRC-OA protects SW1353 cells from ROS-induced cell death. BRC-OA derived from 5 different donors significantly increased cell survival in the presence of H2O2: 56% average cell viability in the presence of BRC-OA vs <10% in wells with H2O2 only (red bar).

Key anti-inflammatory, anti-oxidant and protease inhibiting factors in BRC-OA. FIG. 19 presents a summary of average levels of key factors known for anti-inflammatory, anti-oxidant and protease inhibiting properties (table below). The presence of multiple anti-inflammatory factors in BRC-OA ensures the robust regulation of inflammation. Each factor listed in Table 2 is currently under evaluation for treatment of OA.

TABLE 2
Factors evaluated for treatment of Osteoarthritis

| Factors | Key Activity |
|---|---|
| Soluble TNFRI | Inhibitor of TNF-□ |
| IL-1RA (IL-1 receptor antagonist) | Inhibitor of IL-1 |
| □2-macroglobulin | Pan protease inhibitor |
| TGF-□1 | Anti-inflammatory |
| Adiponectin | Metabolism & immune response |
| | (macrophage polarization into M2 |
| | anti-inflammatory type) regulator |
| Vitamin E (Tocopherol) | Anti-oxidant, anti-inflammatory |

### E. Example 5. Anti-Inflammatory Activity of BRC-OA In Vitro Versus Unprocessed Devitalized Tissue

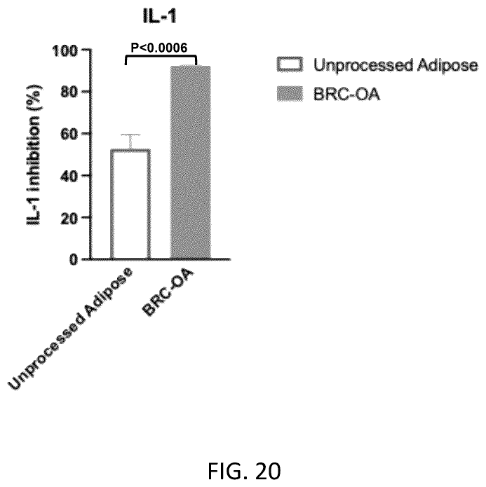

Anti-inflammatory activity of three samples of unprocessed devitalized human adipose tissue and BRC-OA (processed cryopreserved devitalized human adipose tissue) derived from cadaveric donors was tested using THP-1 cells, a model for human monocytes/macrophages as described in Example 4 in the Section “Mechanisms of BRC-OA anti-inflammatory activity”. Results showed that BRC-OA significantly stronger inhibited secretion of IL-1□, an inflammatory cytokine that plays a critical role in development and progression of OA (FIG. 20).

Inhibition of IL-6 and TNF-□ was not significantly different between unprocessed adipose and BRC-OA (Table 3). These results indicate that the developed adipose tissue processing method for manufacturing of BRC-OA selectively eliminates some interfering factors making anti-inflammatory activity stronger than the activity of the native tissue. The literature indicates that highly variable clinical outcomes with the use of autologous adipose to treat OA might be linked to inflammatory properties of the autologous adipose (Cavallo et al. Int. J. Mol. Sci. 2023, 24, 12401). Literature studies show that adipose tissue has detectable levels of IL-1□, IL-6, TNF-□, IFN-□ and other inflammatory cytokines (Table 3) whereas BRC-OA not (FIG. 5).

TABLE 3
Summary of anti-inflammatory properties of native adipose
tissue versus BRC-OA

| | | Literature |
|---|---|---|
| Property | Description | references |
| Inhibition of | BRC-OA > unprocessed | N/A |
| IL-1□ in the | adipose, statistically | |
| THP-1 assay | significant | |
| Inhibition of IL-6 and | IL-6: BRC-OA > | N/A |
| TNF-□ in the | unprocessed adipose, but not | |
| THP-1 assay | statistically significant | |
| | TNF-□: BRC-OA < | |
| | unprocessed adipose, but not | |
| | statistically significant | |
| Inflammatory Cytokines | BRC-OA: Below detectable | Regulski et al., |
| (IL-1□, IL-6, IFN-□, | except IL-6 | Plast Reconstr |
| G-CSF, MIG, TNF-□) | Native Adipose: Detectable | Surg Glob |
| | | Open 2024; |
| | | 12:e6404 |

Abbreviations: G-CSF—Granulocyte Colony Stimulating Factor; IFN—interferon; IL—Interleukin; MIG—Monokine induced by IFN-□; N/A—not applicable; TNF—Tumor Necrosis Factor; N/A—not applicable

Comparison of the anti-inflammatory cytokine profile and the anti-inflammatory activity of BRC-OA versus native adipose tissue in vitro: i) BRC-OA has no detectable inflammatory cytokines except MCP-1, native tissue has detectable cytokines based on data in the literature; ii) Inhibition of IL-1□ (considered one the most critical cytokines in development and progression of OA) by BRC-OA is significantly stronger in comparison to the unprocessed devitalized tissue. It means that the process eliminates interfering factors present in native adipose. These experiments should help to differentiate BRC-OA from the adipose native tissue.

### F. Example 6. Anti-Inflammatory Activity of BRC-OA In Vivo in Naïve Sprague Dawley (SD) Rats After Subcutaneous (SC) Implantation May be Mediated by M2 Anti-Inflammatory Macrophages

The objective of this study was to evaluate anti-inflammatory activity of human adipose xenografts BRC-OA and BRC001 (comprised of BRC-OA tissue and adipose-derived culture expanded SVF cells) after SC implantation in naive SD rats in vivo using Western Blot (WB) and immunohistochemical (IHC) methods. The animal model was as described in Example 3. Briefly, the study was conducted using twelve 4-5 weeks old male SD rats. Animals were randomly divided in 2 groups: Group 1 (n=6), BRC001 (BRC-OA+SVF cells); and Group 2 (n=6), BRC-OA alone. Implantation of human adipose formulations was performed after one week acclimation (Day 0). All rats under general anesthesia received subcutaneously ˜0.5 mL/point of BRC001 or BRC-OA formulations in 4 points on the shaved dorsum (2 on the left and 2 on the right side of the spinal column, total 2 mL) using a 20G needle. Euthanasia and necropsy sample collections from animals in Group 1 and Group 2 (one rat from each group) were performed on days 3, 7, 10, 14, 21, and 28 post-implantations. On euthanasia days, gross examination of animals was performed, then, the skin on the back of each rat was cut open, and the grafts were identified, photographed, dissected out, and weighed. One graft from each animal was fixed in 10% NBF for histological analysis, another graft was collected in a tube containing medium with antibiotics for cell analysis, and the remaining two grafts were flash-frozen on dry ice for biochemical analyses.

WB Analysis: prepared radioimmunoprecipitation assay (RIPA) lysate adipose samples and pre-stained protein ladder standards were loaded onto the 4-12% NuPAGE™ Bis Tris Mini Gel. The gel was run in the MOPS SDS buffer. PAGE and WB were performed according to the manufacturer's recommended protocols. After protein transfer from the gel onto the PVDF WB membrane, the membrane was stained with Ponceau S to confirm equal protein content in each lane. The membrane was probed with mouse anti-rat IL-10 antibody and rabbit anti-CD206 antibody, which cross reacts with mouse, rat, human, and monkey CD206 followed by incubation in the goat anti-mouse (for IL-10) or in anti-rabbit antibody (for CD206) conjugated with HRP. To detect the CD206 and IL-10 protein bands, the membrane was submerged in the ECL solution followed by acquisition of WB images using an imaging device.

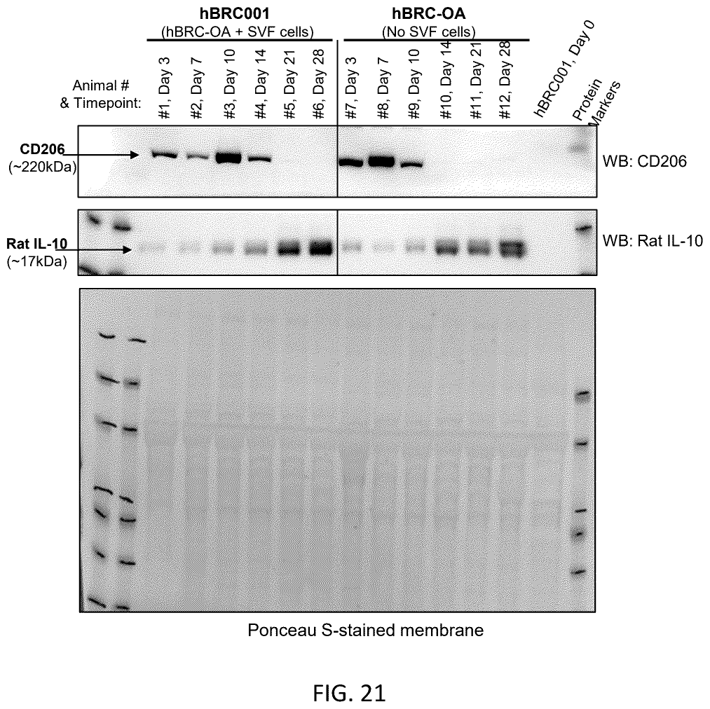

WB analysis demonstrated that hBRC001 and hBRC-OA after SC implantation in rats became populated by M2 macrophages (CD206-positive cells), which are an anti-inflammatory macrophage (FIG. 21, the upper panel). M2 macrophages were detectable at day 3 after hBRC001 and hBRC-OA implantation and persisted for 10-14 days. The presence of M2 macrophages in hBRC001 and hBRC-OA correlates with the detection of rat IL-10, an anti-inflammatory cytokine that M2 macrophages can secrete. In contrast to M2 macrophages, which disappeared from hBRC001 and hBRC-OA after 10-14 days, the amount of rat IL-10 in human adipose formulations increased over time, reaching the highest level at day 28, the last time point in the study (FIG. 21, the middle panel). M2 macrophages (CD206-positive cells) and rat IL-10 were not detectable in hBRC001 prior to implantation (day 0).

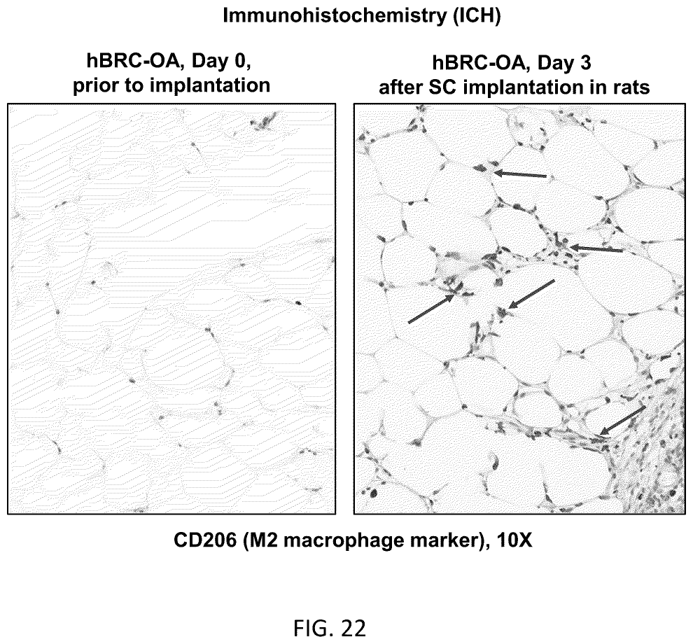

The presence of M2 (anti-inflammatory) macrophages in hBRC001 and hBRC-OA was also evaluated by IHC staining using antibodies against CD206 (1:1,600 dilution, Cell Signaling Technology®, Danvers, MA). All formalin-fixed liver tissue processing and stainings were performed at Histoverv (Germantown, MD). IHC staining shows that as early as on day 3 after SC implantation in rats, hBRC-OA became populated by rat M2 macrophages (FIG. 21, red arrows). FIG. 22 shows that human BRC-OA (labeled “hBRC” on the figure) prior to in vivo implantation had no CD206 positive cells; however, as early as 3 days after implantation in SD rats, human BRC-OA became populated by CD206 marker expressing cells. This result indicated that rat anti-inflammatory M2 macrophages populate BRC-OA. WB analysis of human or rat BRC in Zucker rats 28 days after implantation confirms that in vivo BRC is populated by rat M2 macrophages (FIG. 21). Moreover, the presence of CD206 positive cells correlates with the appearance of IL-10 in the adipose formulations after in vivo implantation (FIG. 21). These results support that in vivo M2 macrophages play a role in BRC-OA anti-inflammatory activity in at the site of implantation.

Anti-inflammatory activity of BRC-OA in vivo in naïve SD rats (with a comparison to BRC001, which composed of BRC-OA+culture expanded MSCs): Rat M2 macrophages populated the implants, which was correlated with rat IL-10 detection in the human implants. There were no differences between devitalized tissue (BRC-OA) and a combination BRC-OA+culture expanded MSCs—concluding that the tissue is the critical component and the presence of viable cells is not required. Inflammatory M1 macrophages are abundant in the OA synovium. M1 macrophages contribute to cartilage damage through the release of pro-inflammatory cytokines such as IL-1β. Thus, polarization of synovial macrophages from M1 (inflammatory) into M2 (anti-inflammatory) would be beneficial for the treatment of OA. These findings suggest that switching macrophage polarization from inflammatory M1 to anti-inflammatory M2 macrophages by BRC-OA might be one of its mechanisms of action.

### G. Example 7. BRC-OA Mobilize Mesenchymal Stem Cells (MSCs) In Vitro and In Vivo BRC-OA Ability to Mobilize MSCs was Evaluated in In Vitro Transwell Chemotaxis Assay

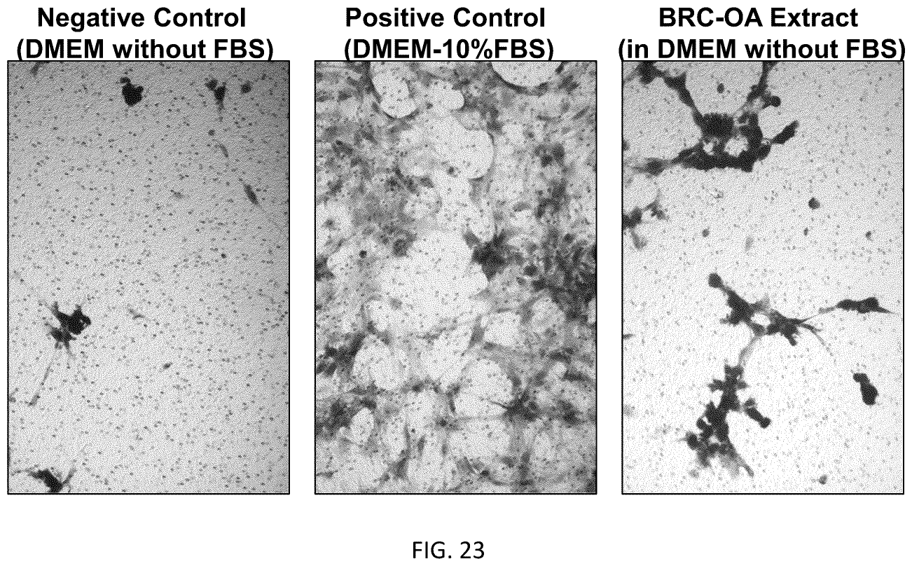

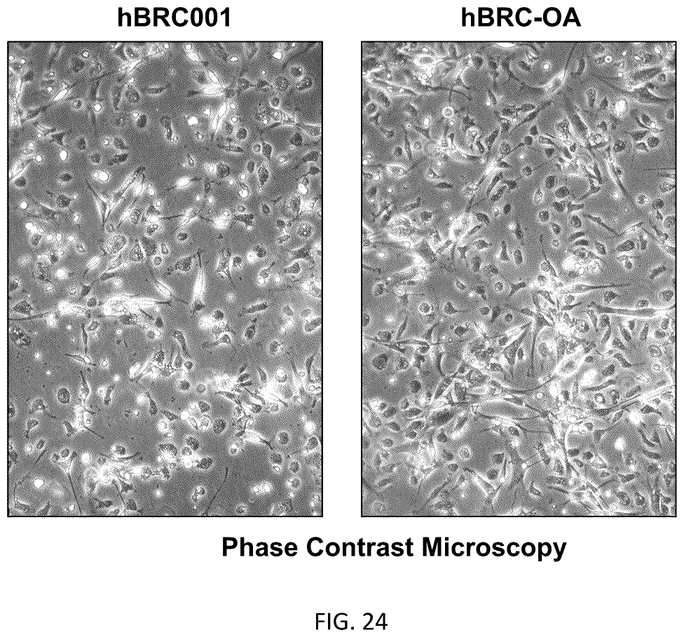

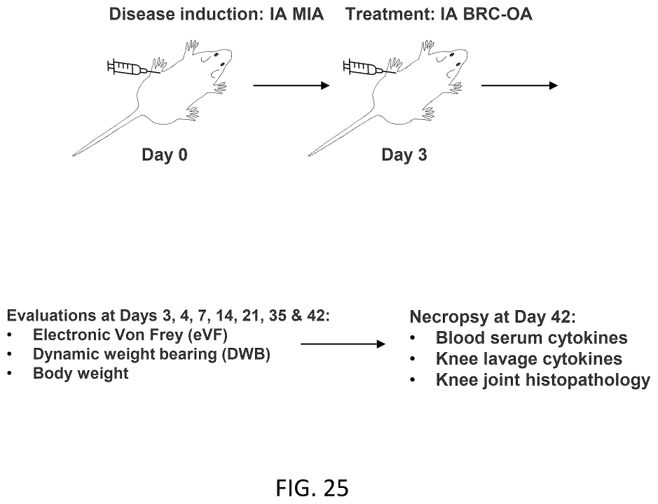

0.6 mL of DMEM without serum (negative control) or DMEM with 10% FBS (positive control) or BRC-OA derived extract in DMEM without FBS were placed in wells of a 24-well plate. Then, adipose-derived culture expanded human MSCs at 50,000 cells/well were place in 0.1 mL DMEM without FBS in an insert of a 0.31-cm² filter with 8-μm pores. The plate was incubated overnight in a CO₂ incubator. After overnight incubation cells from the top of the filters were pipetted out and wiped with a cotton swabs. Cells on the bottom of the filter (migrated cells) were fixed with 0.6 mL mL/well 4% formaldehyde. Fixed cells were stained in 0.6 mL/well 1% gentian violet in 10% ethanol, and then washed 3 times in PBS (1 mL/well). Cell migration was evaluated microscopically. Results show that BRC-OA induces MSC migration in vitro (FIG. 23). These in vitro results are in line with in vivo results. In vivo results demonstrate that after SC implantation in the naïve SD rat model (Described in Example 6) BRC001 and BRC-OA became populated by rat cells. Rat cells were isolated from BRC001 and BRC-OA and expanded ex vivo in culture flasks (FIG. 24). These cells have characteristics of MSCs: they are adhere to plastic, have spindle-like morphology (FIG. 24) and can be differentiated at least into adipocytes (Data not shown). There were no difference between cells isolated from BRC001 and BRC-OA indicating that MSC mobilizing activity is mediated by the tissue, and the presence of viable cells is not critical.

In vitro MSC chemotaxis data show that BRC-OA can attract mesenchymal stem cells (MSCs). MSCs were incubated in a transwell of a 24-well culture plate in the presence of a BRC-OA extract. MSCs migrated through a porous membrane. This in vitro result is in line with in vivo data showing that BRC-OA became populated by host MSCs. Rat cells were isolated from BRC-OA after SC implantation and cultured. These rat cells adhere to plastic, have a spindle-like fibroblast morphology, and can be differentiated into mesenchymal types of cells (at least into adipocytes). It indicates that BRC-OA can mobilize MSCs, which might be beneficial for cartilage repair in OA.

### H. Example 8. BRC-OA Safety and Therapeutic Effects Evaluation in a Rat Model of OA

### 1. Description of the Model.

The study can be conducted using twenty-one 375-400 g male SRG (Rag2 knock-out) Sprague Dawley (SD) rats. On study day 0, animals can be anesthetized, right knees shaved and prepped with 70% EtOH, and then injected with 2 mg MIA into the right knee joint to induce OA. On study day 2, animals can undergo electronic Von Frey (eVF) analysis and then be randomized into groups so the mean eVF force is comparable for all treatment groups. On study day 3, animals can be anesthetized for dosing with 50 □L/joint PBS (placebo control), BRC-OA (4-fold diluted in PBS), or triamcinolone+tramadol (positive control−steroid drug+opioid analgetic). Behavior assessment, eVF, and dynamic weight bearing (DWB) can be performed by a blinded evaluator. Body weights can be collected on days 3, 4, 7, 14, 21, 28, 35 and 42. Post pain testing on day 42, animals can be sacrificed by isoflurane anesthesia followed by bilateral pneumothoracotomy and blood serum collection and knee synovial fluid lavage, and knee joints and patella tendon with synovium can be collected for analyses.

### 2. Dynamic Weight Bearing (DBW) Assessment.

Prior to testing, the force sensor is cleaned lightly with 70% isopropyl alcohol and wiped clean of any debris. The lid is placed on the DWB chamber (BioSeb Cat. #: BIO-DWB-DUAL), and the camera is checked for proper alignment and adjusted as necessary. Rats can be placed into the DWB testing chamber and allowed to move freely for 2-3 minutes. Approximately 1-2 minutes of video can be used for data analysis. Animals can be assessed for amount of weight placed on the left and right hind paw (in grams) and can be reported as grams of difference between each foot.

3. Electronic Von Frey (eVF) Testing.

Rats are habituated to an animal colony for one week and handled four times for five minutes each after the week of habituation. Rats are habituated to the testing rack three times during this process. Rats can be tested using an Almemo 2490 eVF testing device with an 800 g probe handle. Prior to each testing session, a rigid tip can be attached to the black cone of the transducer with a slight twist while holding the cone to prevent pushing too firmly and damaging the instrument. The 8.1 GR calibrator is placed on the transducer, which should measure between 8 and 8.3 g. After these steps are completed, the instrument is ready for testing. After a minimum of 5 minutes of acclimation, the instrument can be zeroed out, and then gentle pressure can be applied with the rigid tip to the testing area (between the foot pads/palm of the injected hind limb) at a 90° angle. Constant pressure can be applied until the rat raises its foot in response to the stimulus. If it is unclear if the rat moved, rather than a true response, the rat can be retested after a 2-3 min break. Each rat can be tested 3 times with a minimum of 8 minutes between testing events. The force applied for each response can then be averaged for each animal.

### 4. Histology.

Following 4-6 days in 5% formic acid decalcifier, the operated joints are cut into 2 approximately equal halves in the frontal plane and embedded in paraffin. Sections are cut from each right knee and stained with toluidine blue. If additional stains are requested, additional blank slides can be cut after the toluidine blue section. Stained tissue sections can be assessed by a blinded pathologist.

### 5. Multiplex Analysis for 5 Rat Inflammatory Cytokines in Rat Serum and Knee Lavage.

Collected 12 knee joint lavage samples were tested neat without dilution. Collected 21 blood serum samples were tested at 10-fold dilution. A custom ProcartaPlex™ Rat Cytokine Panel, 5-plex (Rat IL-10, IL-6, IL-1□, TNF-□□ and MCP-1) (ThermoFisher) was used to measure levels of cytokines in samples according to the manufacturer's protocols using the Luminex® FLEXMAP 3D® instrument. Multiplex results were calculated using the xPONENT® software for the Luminex® FLEXMAP 3D®.

### 6. Statistical Analysis.

Data can be analyzed using a Student's t-test or Mann-Whitney U test (non-parametric). If appropriate, data will be further analyzed across all groups using a one-way analysis of variance (1-way ANOVA) or Kruskal-Wallis test (non-parametric) along with the appropriate multiple comparison post-test. Significance for all tests is set at p≤0.05.

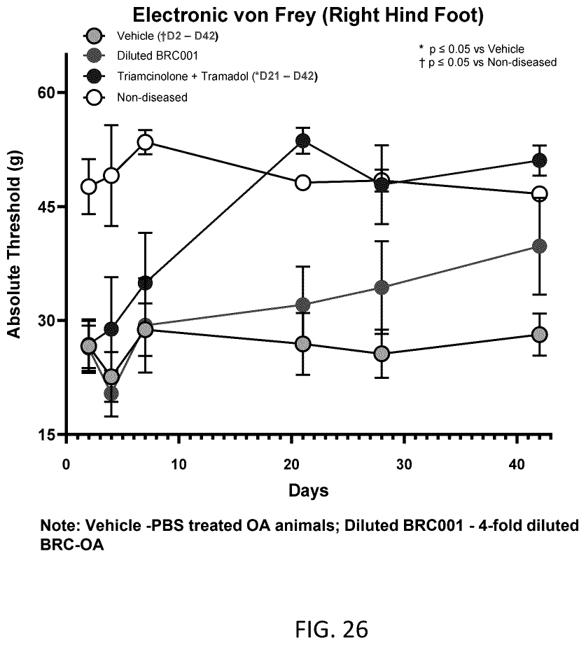

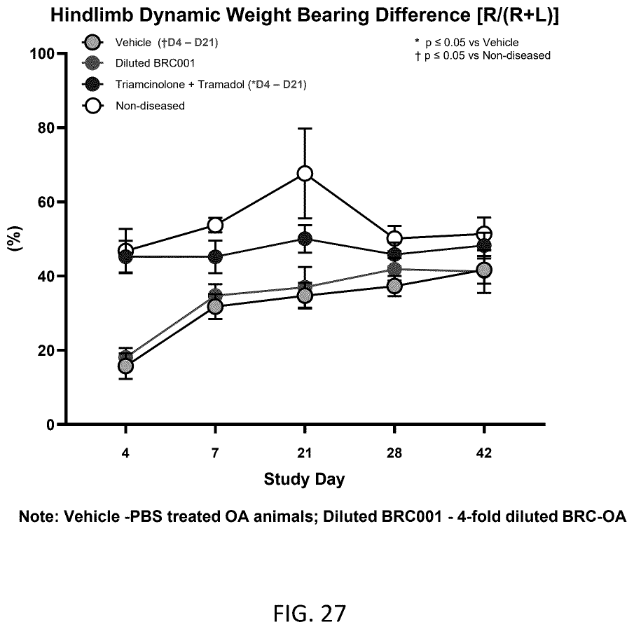

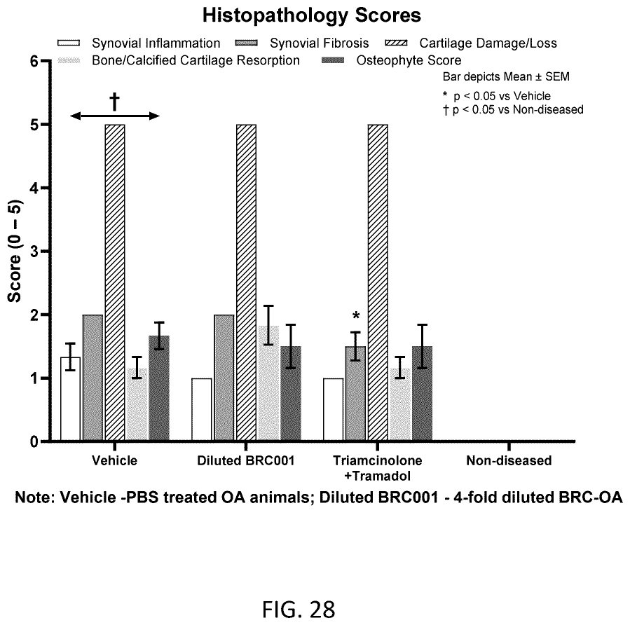

Results demonstrated that BRC-OA IA injections were well tolerated without negative impact on animal's health and behavior. There were no differences in body weight between groups. Electronic von Frey test demonstrated that compared to non-diseased controls, diseased controls showed decreased absolute force threshold (FIG. 26) from day 2 through day 42. Relative to diseased controls, rats treated with BRC-OA had increased absolute force threshold from day 21 through d24, but it did not reach statistical significance. Relative to diseased controls, rats treated with triamcinolone and Tramadol had increased absolute force threshold from day 21 through day 42. Compared to non-diseased controls, diseased controls showed decreased hindlimb DWB percentage (FIG. 27) from day 4 through day 21. Relative to diseased controls, rats treated with triamcinolone and Tramadol had increased hindlimb DWB percentage from day 4 through day 21. Diseased controls had significantly increased histopathologic scores of synovial inflammation, synovial fibrosis, cartilage damage/loss, bone/calcified cartilage resorption, and osteophytes compared to non-diseased controls (FIG. 28). Rats treated with BRC-OA had reductions (≥20%) in synovial inflammation were apparent but did not reach significance. Due to differences between species it is expected that human growth factors and cytokines will have weaker biological activities in rats. Rats treated with triamcinolone and Tramadol had significantly decreased synovial fibrosis compared to diseased controls. Reduction (≥20%) in synovial inflammation was apparent but did not reach significance. Thus, BRC-OA had effects similar to the positive control used in the study.

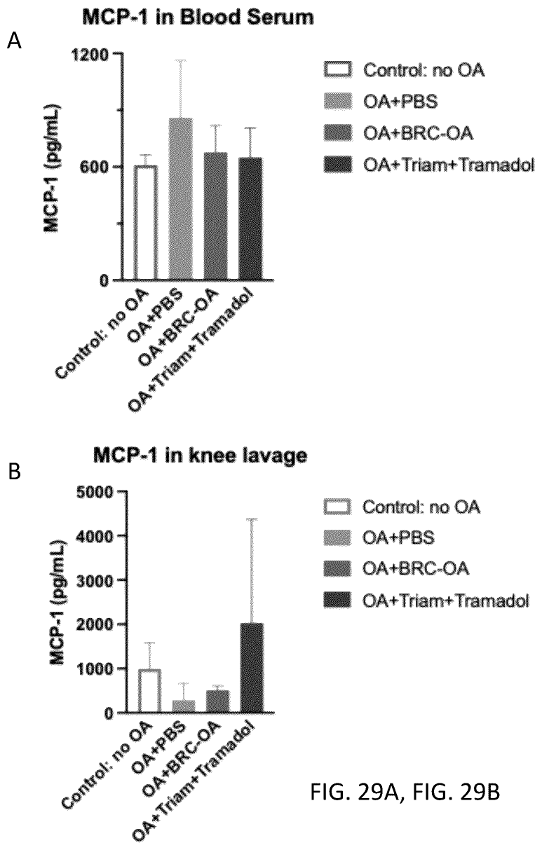

Testing of blood serum and knee lavage for 5 inflammatory cytokines showed that only monocyte chemoattractant protein 1 (MCP-1) was detectable. Interleukin-10 (IL-10), IL-6, IL-1□□ and TNF-□ were below detectable levels in both blood serum and knee joints lavage. In blood serum MCP-1 levels were not different from MCP-1 levels in serum of healthy animals and lower in comparison to PBS-treated animals with OA indicating that BRC-OA has anti-inflammatory effect (FIG. 29A). In the knee lavage the level of MCP-1 after BRC-OA treatment was not different from the level of MCP-1 in knee lavage of healthy animals. However, in PBS treated animals the level of MCP-1 was significantly lower than the MCP-1 level in knee lavage from healthy animals (FIG. 29B). This can be explained by a low volume of synovial fluid in the joint at the end stage of OA, which is day 42 in the rat MIA OA model.

Testing of BRC-OA in a rat MIA OA model shows that it is safe with an efficacy trend—the decrease in synovial inflammation (by histopathology) and pain (by the von Frey behavioral test), but did not reach statistical significance (probably due to weaker biological activity of human growth factors and cytokines in rats). The effects of BRC-OA was comparable with the effects of the positive control (steroid+opioid).

Those skilled in the art will recognize, or be able to ascertain using no more than routine experimentation, many equivalents to the specific embodiments of the method and compositions described herein. Such equivalents are intended to be encompassed by the following claims.

1. A method of treating a subject having osteoarthritis comprising administering to the subject a composition, wherein the composition comprises an adipose matrix comprising devitalized and partially delipidized adipose tissue.

2. The method of claim 1, wherein the partially delipidized adipose tissue comprises at least 20% of the original lipids.

3. The method of claim 1, wherein the devitalized and partially delipidized adipose tissue is minced.

4. The method of claim 3, wherein the minced adipose tissue comprises pieces of adipose tissue 1 mm in diameter.

5. (canceled)

6. The method of claim 1, wherein the composition or adipose matrix is cryopreserved, has been previously cryopreserved, is lyophilized or has been previously lyophilized.

7. (canceled)

8. The method of claim 1, wherein the composition or adipose matrix has not been cryopreserved or lyophilized

9. (canceled)

10. The method of claim 1, wherein the composition further comprises mesenchymal stem cells (MSCs).

11. The method of claim 10, wherein the MSCs are adipogenic differentiated MSCs.

12. The method of claim 10, wherein the MSCs are allogeneic or autologous to the adipose matrix.

13. (canceled)

14. The method of claim 10, wherein the MSCs are present in an amount of 5×10⁵ to 50×10⁶ MSCs per 1 milliliter of devitalized and partially delipidized adipose tissue.

15. The method of claim 10, wherein the MSCs are culturally expanded.

16. The method of claim 1, wherein the composition is administered intra-articularly.

17. The method of claim 1, wherein the adipose matrix is derived from the adipose of the subject who is being administered the composition or from the adipose of a subject different from the subject who is being administered the composition.

18. (canceled)

19. The method of claim 1, wherein the adipose matrix is derived from a cadaver or lipoaspirate.

20. The method of claim 1, wherein the composition further comprises a pharmaceutically acceptable carrier.

21. The methods of claim 1, wherein devitalized adipose tissue comprises <5% viable cells.

22.-43. (canceled)

44. A method of inhibiting inflammatory cytokines in a subject having osteoarthritis comprising administering to the subject a composition, wherein the composition comprises devitalized and partially delipidized adipose tissue.

45. (canceled)

46. (canceled)

47. A method of inhibiting MMP-13 in a subject having osteoarthritis comprising administering to the subject a composition, wherein the composition comprises devitalized and partially delipidized adipose tissue.

48. (canceled)

49. The method of claim 1, where treating a subject having osteoarthritis comprises treating or ameliorating a symptom of osteoarthritis in the subject.

50. The method of claim 49, wherein the symptom of osteoarthritis is inflammation, pain, and/or poor quality of life.
